(19) United States
(12) Patent Application Publication
(10) Pub. No.: US 2024/0366658 A1
(43) Pub. Date: Nov. 7, 2024

(54) DEODORANT COMPOSITIONS CONTAINING ZINC AND ERYTHRITOL

(71) Applicant: InnoMed Technologies, Inc., Encino, CA (US)

(72) Inventor: John Joseph, Westlake Village, CA (US)

(73) Assignee: InnoMed Technologies, Inc., Encino, CA (US)

(21) Appl. No.: 18/772,268

(22) Filed: Jul. 15, 2024

Related U.S. Application Data
(63) Continuation-in-part of application No. 18/306,099, filed on Apr. 24, 2023.
Application No. 18/306,099 is a continuation-in-part of application No. 18/075,158, filed on Dec. 5, 2022.
Application No. 18/075,158 is a continuation-in-part of application No. 17/673,532, filed on Feb. 16, 2022, now Pat. No. 11,517,578.
(60) Provisional application No. 63/227,322, filed on Jul. 29, 2021.

(51) Int. Cl. A61K 33/30; A61K 8/27; A61K 8/34; A61K 9/00; A61K 31/047; A61K 31/14; A61L 2/18; A61L 2/232; A61L 101/06; A61L 101/30; A61L 101/34; A61P 31/04; A61P 31/10; A61Q 15/00; A61Q 17/00

(52) U.S. Cl. CPC A61K 33/30; A61K 8/27; A61K 8/345; A61K 9/0014; A61K 31/047; A61K 31/14; A61L 2/18; A61L 2/232; A61P 31/04; A61P 31/10; A61Q 15/00; A61Q 17/005; A61L 2101/06; A61L 2101/30; A61L 2101/34; A61L 2202/24

(57) ABSTRACT

Embodiments include formulations for topical administration of sugar alcohol to treat a skin condition such as athlete's foot, a yeast infection, jock itch (Tinea cruris), a toe nail infection, diaper rash, diaper candidiasis, ringworm (Tinea capitis), impetigo, erythrasma, pitted keratolysis, trichomycosis and sphingomonas paucimobilis. The formulation can include a moisturizer, an emollient, a sugar alcohol and zinc. In aspects, the sugar alcohol is erythritol with zinc chloride at a molar ratio of about 3:1. Embodiments also include erythritol-zinc antimicrobial coatings for personal use products.

### RELATED APPLICATIONS

The present application is a continuation-in-part of U.S. application Ser. No. 18/306,099 filed Apr. 24, 2023 which is a continuation-in-part of U.S. application Ser. No. 18/075,158 filed Dec. 5, 2022 which is a continuation-in-part of U.S. application Ser. No. 17/673,532 filed Feb. 16, 2022, (now U.S. Pat. No. 11,517,578) which claims priority to U.S. Provisional Application No. 63/227,332 filed Jul. 29, 2021, the contents of which are incorporated herein by reference.

### FIELD OF THE INVENTION

The invention relates generally to dermatologic methods, and more specifically to methods and formulations of transdermal administration of erythritol and zinc to treat ailments and improve the appearance of skin.

### BACKGROUND

Skin care includes a range of practices that support skin integrity, enhance its appearance and relieve skin conditions. The practices can include nutrition, avoidance of excessive sun exposure and use of emollients. Practices that enhance appearance include the use of cosmetics, botulinum, exfoliation, fillers, laser resurfacing, microdermabrasion, peels, retinol therapy and ultrasonic skin treatment. Skin care is generally a routine daily procedure with a primary goal of improving one's appearance.

Acne, also known as acne vulgaris, is a long-term skin condition that occurs when dead skin cells and oil from the skin clog hair follicles. Signs and symptoms of acne include blackheads or whiteheads, pimples, oily skin and scarring in some patients. Acne mainly affects areas of the skin that have a high number of oil glands such as the face, upper part of the chest and back. Although generally considered a cosmetic concern, acne can lead to anxiety, reduced self-esteem and depression.

Acne commonly occurs in adolescence and affects an estimated 80-90% of teenagers in the Western world. Although acne becomes less common in adulthood, it persists in nearly half of affected people into their twenties and thirties, and a smaller group continues to have difficulties in their forties. Risk factors for the development of acne, other than genetics, have not been conclusively identified. Possible secondary contributors include hormones, infections, diet, and stress. The anaerobic bacterial species Cutibacterium acnes (C. acnes) contributes to the development of acne, but its exact role is not well understood. There are specific sub-strains of C. acnes associated with normal skin and others with moderate or severe inflammatory acne. It is unclear whether these undesirable strains evolve on-site or are acquired, or possibly both depending on the person.

Many different treatments exist for acne. These include alpha hydroxy acid, anti-androgen medications, antibiotics, antiseborrheic medications, azelaic acid, benzoyl peroxide, hormonal treatments, steroids, keratolytic soaps, nicotinamide, retinoids and salicylic acid. Acne treatments work in at least four different ways including: reducing inflammation, hormonal manipulation, killing C. acnes, and normalizing skin cell shedding and sebum production in the pore to prevent blockage. Typical treatments include topical therapies such as antibiotics, benzoyl peroxide, and retinoids, and systemic therapies, including antibiotics, hormonal agents, and oral retinoids.

Antibiotics can be applied to the skin (i.e., dermal application) or taken orally. They work by killing C. acnes and reducing inflammation. Although multiple guidelines call for healthcare providers to reduce the rates of prescribed oral antibiotics, many providers do not follow this guidance. Oral antibiotics remain the most commonly prescribed systemic therapy for acne. Widespread broad-spectrum antibiotic overuse for acne has led to higher rates of antibiotic-resistant C. acnes strains worldwide, especially to the commonly used tetracycline (e.g., doxycycline) and macrolide antibiotics (e.g., topical erythromycin).

Commonly used antibiotics include clindamycin, erythromycin, metronidazole, sulfacetamide, and tetracyclines (e.g., doxycycline or minocycline). Antibiotics applied to the skin are typically used for mild to moderately severe acne. Oral antibiotics are generally more effective than topical antibiotics and produce faster resolution of inflammatory acne lesions than topical applications.

Antibiotic treatments for acne have shortcomings. Antibiotics, especially broad-spectrum antibiotics, can present side effects including nausea, indigestion, vomiting, diarrhea, bloating and stomach cramping. Oral antibiotics are not recommended for longer than three months. Longer durations of antibiotic treatment are associated with the development of antibiotic resistance. And while antibiotics reduce bacteria and inflammation, they do nothing to reduce pore blockages and the formation of microcomedones (the tiny beginnings of a pimple under the skin).

A biofilm is a thin film of mucus created by a colony of bacteria and other microorganisms. A biofilm can be further described as any syntrophic consortium of microorganisms in which cells stick to each other and often also to a surface. These adherent cells become embedded within a slimy extracellular matrix that is composed of extracellular polymeric substances (EPSs). The cells within the biofilm produce the EPS components, which are typically a polymeric conglomeration of extracellular polysaccharides, proteins, lipids and DNA. Because they have three-dimensional structure and represent a community lifestyle for microorganisms, they have been metaphorically described as “cities for microbes”.

Biofilms may form on living or non-living surfaces and can be prevalent in natural, industrial, and hospital settings. Biofilms have been implicated in acne vulgaris patients when conventional treatments are ineffective, begging the need for more effective targeted therapies directed against biofilm formation. Biofilms also present risks of infection in clinical settings. They can often proliferate on surfaces of medical devices and spread to a patient or practitioner upon exposure. For example, biofilm present on implantable devices can lead to dangerous and recurring infections that can jeopardize the health of subjected patients.

There is a need for more effective reduction, negation, and elimination of biofilm. The compositions and methods described herein provide safe and effective means of penetrating biofilm and ameliorating bacterial growth. Accordingly, aspects of the invention include formulations and methods that utilize erythritol and zinc for reducing, negating and eliminating the formation of biofilm to treat skin conditions and improve the appearance of one's skin.

### SUMMARY OF THE INVENTION

Aspects of the present disclosure teach certain benefits in construction and use which give rise to the exemplary advantages described below.

One embodiment is a composition and method for ameliorating, removing and/or penetrating biofilm present on a medical device. In aspects, the composition is an antimicrobial coating comprising erythritol and zinc, its method of use described by the disclosure herein. In aspects, the composition is used on a medical device (e.g., a catheter or cannula). In yet other aspects, the composition is used to reduce, remove or penetrate biofilm on a comfort apparatus utilized by a patient, such as a bed, bedding, cantilevered support bars, hospital bed rails, and any surface in which a biofilm may develop.

One embodiment is a method of treating acne vulgaris (i.e., acne). The treatment can reduce the size and/or number of comedones, papules and/or pustules. The method includes topical administration of a therapeutic amount of a sugar alcohol and zinc. The sugar alcohol can be erythritol, xylitol, mannitol, sorbitol, ethylene glycol, glycerol, threitol, arabitol, xylitol, ribitol, mannitol, galactitol, fucitol, iditol, inositol, volemitol, isomalt, maltitol, lactitol, maltotriitol, maltotetraitol and/or polyglycitol. In aspects, the acne is facial acne. In aspects, the acne is body acne.

The method can also include administration of another agent or medicament to treat acne such as benzoyl peroxide, a retinoid, a steroid, an antibiotic, azelaic acid, salicylic acid, dapsone, a contraceptive, an anti-androgen agent or isotretinoin to the subject.

In one embodiment, the sugar alcohol is erythritol and the erythritol and zinc are provided in a formulation at a molar ratio of about 3:1. In another embodiment, the sugar alcohol is xylitol and the xylitol and zinc are provided at a molar ratio of about 3:1. In other embodiments, the sugar alcohol is mannitol and the mannitol and zinc are provided at a molar ratio of 3:1. In other embodiments, the sugar alcohol may be any known sugar alcohol in the art, and may be provided with zinc or zinc chloride in a molar ratio of about 1:1, about 1:2, about 1:3, about 2:1, about 2.5:1, about 3:1, about 3.25:1, about 3.5:1, about 3.75:1, about 4:1, about 5:1, about 6:1, about 7:1, about 8:1, about 9:1, about 10:1, about 20:1, about 50:1, about 100:1.

In another embodiment, the sugar alcohol and zinc are co-administered to a subject with a transdermal penetrant. The formulation can also include a humectant, an emulsifier and/or an emollient.

In one embodiment, the method includes administration of one or more additional agents such as benzoyl peroxide, a retinoid, a steroid, an antibiotic, azelaic acid, salicylic acid, dapsone, a contraceptive, an anti-androgen agent and isotretinoin.

The topical formulation can have a pH of 9-11. Alternatively, it can have a pH of 7-10.5. In one embodiment, the topical formulation can have a pH exceeding the bounds of this range by about 5%. In other embodiments, the topical formulation can have a pH exceeding the bounds of the aforementioned range by about 6%, by about 7%, by about 10%, by about 15% by about 20%, by about 25%, by about 30%, or more.

Another embodiment is a method of treating a skin condition. The method can include a step of administering a medicament to an area of skin of a subject. The medicament can include erythritol and zinc chloride. The erythritol and zinc chloride can be at a molar ratio of about 3:1. The skin condition can be acne, cellulitis, erysipelas, bacterial folliculitis, hot tub folliculitis, furuncles, carbuncles, impetigo, erythrasma and/or MRSA skin infection. In aspects, the skin condition is caused by yeast or fungus.

In embodiments, the compositions described herein target both bacteria and yeast/fungi. Alternatively, the compositions can target one or bacteria and yeast/fungi. In embodiments, the compositions and methods include a medicament of erythritol and benzethonium chloride (without Zinc).

In one embodiment, multiple agents are administered together to treat a skin condition. The multiple active agents can act synergistically with one another.

Another embodiment is an antimicrobial coating for a surface of a substrate such as a medical device. In aspects, the medical device is intended for invasive use.

The compositions (i.e., formulations and coating solutions) can include a sugar alcohol and zinc. In aspects, the sugar alcohol is erythritol. In aspects, the erythritol and zinc are present in a molar ratio of about 3:1. These agents can act synergistically with one another.

In aspects, the antimicrobial coating is provided as a solution that includes 6.6 mM zinc chloride and 19.8 mM erythritol. The solution can be applied to a surface of a medical device. Thereafter, a coating is produced after a drying step. The solution can also include a solvent, inert ingredients, a lubricant and additional substances with antibiotic effects. In aspects, the coating reduces or prevents growth of yeast/fungus.

In aspects, the antimicrobial coating is formulated for delayed release. In aspects, the antimicrobial coating includes one or more adhesive agents to improve qualities of coating and release. In aspects, the antimicrobial coating includes one or more additional disinfectants or antiseptics (e.g., benzethonium chloride).

In aspects, the antimicrobial coating also includes one or more additional antibacterial agents such as a silver compound or an antibiotic.

In aspects, the antimicrobial coating includes emollient solvents to improve the lubricity of the surface. Certain natural oils (e.g., flax seed oil, grape seed oil, avocado oil, or cranberry oil) can be included for improved lubricity and for anti-inflammatory effects.

Accordingly, embodiments include antibacterial compositions that can be used to coat medical devices to form a stable, effective and lubricious antimicrobial coating. In aspects, the compositions reduce or prevent growth of yeast/fungus.

The medical device can be, for example, a catheter and tubing (e.g., a urinary catheter, central venous catheter, endotracheal tube, cannulae, etc.). The catheter can be, for example, an indwelling catheter, a condom catheter or an intermittent self-catheter. Such devices can be made of material such as latex, silicone or a plastic.

In aspects, the medical device is a long-standing indwelling catheter such as a feeding tube. Accordingly, embodiments include methods of preventing or ameliorating infection incident to catheterization.

In aspects, the medical device is an intravenous (IV) catheter, a central line catheter (e.g., PICC, CVC or port catheter). In aspects, the catheter is a component used for infusion of a chemotherapeutic agent. In aspects, the medical device is a standard (or “peripheral”) IV line.

Another embodiment is a method of applying an antibacterial coating to a surface of a medical device. Another embodiment is a method of cleaning a substrate such as a medical device using a solution of erythritol and zinc (present in a molar ratio of about 3:1).

In aspects, the solution of erythritol and zinc also includes a lubricious material (e.g., a hydrogel). This lubricious material can form a coating on a medical device which prevent and/or ameliorate the formation of biofilm its surface. In aspects, the solution includes one or more additional disinfectants or antiseptics (e.g., benzethonium chloride).

Embodiments also include methods of administering a formulation that contains erythritol and zinc. In aspects, the methods can include a step of microdermabrasion or exfoliation. In aspects, the methods can include a step of dermabrasion. The methods can also include a step of cleaning an area of skin after exfoliation (e.g., via suction massage).

In aspects, the methods can include administration of the formulation simultaneously with exfoliation, microdermabrasion or hydradermabrasion. In aspects, the methods can include administration of the formulation subsequent to an exfoliation, microdermabrasion or hydradermabrasion treatment.

In aspects, the methods can include a step of applying linoleic acid to the area of skin affected by the skin condition. The linoleic acid can be applied after applying the topical formulation to skin.

In aspects, a device (e.g., a hydradermabrasion or HydraFacial™ device) is used to disrupt the skin surface (i.e., via microdermabrasion), apply suction massage and administer a compound of erythritol and zinc to the skin. In aspects, the steps are carried out simultaneously.

Embodiments include a method of treating a skin condition that includes steps of (a) exfoliating an area of skin affected by the skin condition, (b) cleansing the area of skin with suction, and (c) applying a topical formulation to the area of skin. The topical formulation can include erythritol and zinc at a molar ratio of about 3:1.

Embodiments also include a method of improving appearance of an area of skin that includes steps of: (a) exfoliating the area of skin, (b) cleansing the area of skin with suction and (c) applying a topical formulation to the area of skin. The topical formulation can include erythritol and zinc at a molar ratio of about 3:1. The improved appearance of skin can include, for example: (a) thickening keratinous tissue, (b) preventing and/or retarding the appearance of spider veins and/or red blotchiness, (c) preventing and/or retarding the appearance of dark circles, (d) preventing and/or retarding sallowness and/or sagging, (e) softening and/or smoothing skin, (f) preventing and/or relieving itch of human skin, (g) improving skin texture (e.g., wrinkles and fine lines) and/or (h) improving skin color. Inflammation (as well as the appearance of inflammation) can also be reduced.

In aspects, the formulation of erythritol and zinc is administered with additional agents, for example, a cosmeceutical. In aspects, the formulation of erythritol and zinc is administered with a chemical permeation enhancer or transdermal penetrant.

In aspects, the formulation of erythritol and zinc also includes one or more disinfectants, antiseptics, cleansing ingredients, moisturizing ingredients, emollients, humectants, antioxidants, serums and anti-inflammatory agents. In aspects, the formulation of erythritol and zinc also includes one or more of benzoyl peroxide, a retinoid, a steroid, an antibiotic, azelaic acid, salicylic acid, dapsone, a contraceptive, an anti-androgen agent and isotretinoin.

Embodiments include a dermal filler that includes erythritol and zinc at a molar ratio of about 3:1. Aspects include methods of treating a skin condition that includes steps of (a) cleaning/exfoliating an area of skin, (b) applying an anesthetic, and (c) injecting a dermal filler into the area of skin. The dermal filler can include hyaluronic acid (or other agent to add volume to the skin) and erythritol and zinc at a molar ratio of about 3:1.

In embodiments, the formulation of erythritol and zinc described herein reduces the incidence of infection associated with a medical procedure or a cosmetic procedure.

In embodiments, the formulation of erythritol and zinc described herein reduces the incidence of granuloma formation associated with a medical procedure or a cosmetic procedure.

In embodiments, the formulation of erythritol and zinc described herein reduces the incidence of infection associated with implantation of a medical device or a cosmetic device.

In embodiments, the formulation of erythritol and zinc described herein reduces the incidence of granuloma formation associated with implantation of a medical device or a cosmetic device.

Another embodiment is an eye-drop formulation that contains zinc and erythritol. The formulation can be used to treat an eye infection by disrupting/preventing biofilm on the surface of the eyes. The solution can be applied directly to the eyes of a patient (i.e., as an eye drop solution).

Another embodiment is a method of treating an eye infection using a solution of zinc and erythritol. The solution can include erythritol and zinc in a molar ratio of about 3:1. The solution can also be used prophylactically (e.g., on a subject who is at risk of eye infection).

In aspects, the eye drop solution includes about 6.6 mM zinc chloride and about 19.8 mM erythritol in water or saline solution. The solution can also include trace amounts of salt and boric acid. The solution can also include tetrahydrozoline HCl (about 0.05% w/w).

In aspects, the eye drop solution includes one or more inactive ingredients such as ascorbic acid, boric acid, dextrose, glycerin, glycine, magnesium chloride, polixetonium chloride, potassium chloride, sodium borate, sodium citrate, sodium lactate and sodium phosphate.

Embodiments include methods of using zinc-erythritol to reduce risk of infection associated with a medical procedure.

In embodiments, the compositions and methods described herein are used to ameliorate, prevent or treat fungal or yeast growth. In aspects, the compositions and methods can treat a fungal or yeast infection (e.g., athlete's foot, yeast infection, jock itch/tinea cruris, toe nail infection, diaper rash or ringworm. In aspects, the compositions includes about 0.1 to 0.3% w/w benzethonium chloride.

In embodiments, the compositions described herein are formulated for use to apply to the skin as an odor suppressing agent (e.g., in a lotion, cleaning solution, soap or deodorant). In aspects, the compositions reduce body odor on the subject. In aspects, the compositions act as sanitizing agents.

In embodiments, the compositions described herein are formulated as a douche. In aspects, the compositions and methods can treat and/or prevent a yeast infection.

In embodiments, the compositions described herein are formulated for use of feminine hygiene or personal care products (i.e., sanitary pads or tampons). In aspects, the compositions and methods can treat and/or prevent bacterial growth and/or yeast growth. In aspects, the compositions and methods can prevent and/or treat toxic shock syndrome. In aspects, the compositions and methods can prevent and/or treat bacterial vaginosis.

In embodiments, the compositions described herein are formulated for use on disinfecting wipes. In aspects, the disinfecting wipes are intended for skin cleansing and/or facial cleansing. In aspects, the disinfecting wipes are intended for use with infants (e.g., diaper changes and related use).

In embodiments, the compositions described herein are formulated for use on wet wipes. In aspects, the disinfecting wipes are intended for skin cleansing and/or facial cleansing. In aspects, the disinfecting wipes are intended for use with infants (e.g., diaper changes and related use).

In embodiments, the compositions described herein are formulated for use in shampoo. In aspects, the body wash can reduce bacterial and/or fungal growth. In aspects, the shampoo can reduce/prevent dandruff.

In embodiments, the compositions described herein are formulated for use in a body wash. In aspects, the body wash reduces bacterial and/or fungal growth. In aspects, the body wash reduces/prevents body odor. In aspects, the body odor is one or more of Eccrine bromhidrosis, Apocrine bromhidrosis and Exogenous.

In embodiments, the compositions described herein are formulated for use in a deodorant (e.g., a spray, cream or roll on). In aspects, the deodorant reduces bacterial and/or fungal growth. In aspects, the deodorant reduces/prevents body odor. In aspects, the deodorant also has an antiperspirant (e.g., aluminium chlorohydrate or aluminium-zirconium tetrachlorohydrate gly).

Embodiments also include methods of preventing and/or treating skin conditions such as folliculitis. In aspects, the skin conditions are caused by bacteria. In aspects, the skin conditions are caused by yeast.

Embodiments also include methods of preventing and/or treating skin and scalp conditions (e.g., dandruff). In aspects, the skin and scalp conditions are caused by bacteria. In aspects, the skin and scalp conditions are caused by yeast.

In embodiments, the compositions described herein are used in a barrier cream (e.g., diaper cream) to prevent and/or treat an infection.

Also included herein are formulations that include erythritol, zinc and benzethonium chloride for use as sanitizers, antiseptics, deodorants, antibacterials and antifungals. The formulations can be applied to surfaces of objects and/or the skin to prevent, reduce the incidence or treat infection.

Other features and advantages of aspects of the present invention will become apparent from the following more detailed description, which illustrates, by way of example, the principles of aspects of the invention.

### Definitions

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “subject” or “patient” refers to any single animal, more preferably a mammal (including such non-human animals as, for example, dogs, cats, horses, rabbits, zoo animals, cows, pigs, sheep, and non-human primates) for which treatment is desired.

The term “medicament,” “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

The term “disinfectant” refers to a chemical substance or compound used to inactivate or destroy microorganisms on inert surfaces. Disinfection does not necessarily kill all microorganisms, especially resistant bacterial spores; it is less effective than sterilization, which is an extreme physical or chemical process that kills all types of life. Disinfectants are generally distinguished from other antimicrobial agents such as antibiotics, which destroy microorganisms within the body, and antiseptics, which destroy microorganisms on living tissue. Disinfectants are also different from biocides—the latter are intended to destroy all forms of life, not just microorganisms. Disinfectants work by destroying the cell wall of microbes or interfering with their metabolism. It is also a form of decontamination and can be defined as the process whereby physical or chemical methods are used to reduce the amount of pathogenic microorganisms on a surface. Disinfectants can also be used to destroy microorganisms on the skin and mucous membrane, as in the medical dictionary historically the word simply meant that it destroys microbes. Sanitizers are substances that simultaneously clean and disinfect. Disinfectants kill more germs than sanitizers. Disinfectants are frequently used in hospitals, dental surgeries, kitchens, and bathrooms to kill infectious organisms. Sanitizers are mild compared to disinfectants and are used majorly to clean things that are in human contact whereas disinfectants are concentrated and are used to clean surfaces like floors and building premises.

The term “antiseptic” refers to an antimicrobial substance or compound that is applied to living tissue to reduce the possibility of sepsis, infection or putrefaction. Antiseptics are generally distinguished from antibiotics by the latter's ability to safely destroy bacteria within the body, and from disinfectants, which destroy microorganisms found on non-living objects. Antibacterials include antiseptics that have the proven ability to act against bacteria. Microbicides which destroy virus particles are called viricides or antivirals. Antifungals, also known as antimycotics, are pharmaceutical fungicides used to treat and prevent mycosis (fungal infection).

In an embodiment, a “pharmaceutical composition” is intended to include the combination of an active agent with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed. The pharmaceutical composition may be a coating, a powder, a compound, a time-release agent, a paste, a pill, a tablet, a chewable, a dragee, a pellet, a granulate, a liquid, an aerosol, an injectable, a suppository, a dissolvable tablet, or any other known formulation for delivery. The pharmaceutical composition may be contained within a vial, a blister pack, a capsule, a cartridge, a carpule, a reservoir, a container, or within any form of transdermal tape, applicator, or any needled apparatus. Other known formulations and methods of delivery that are known in the art are inherently contemplated by the disclosure of the present invention.

In an embodiment, as used herein, the terms “treating,” “treatment” and the like are used herein to mean obtaining a desired pharmacologic and/or physiologic effect. The effect may be prophylactic in terms of completely or partially preventing a disorder or sign or symptom or spread of infection thereof, and/or may be therapeutic in terms of amelioration of the symptoms of the disease or spread of infection, or a partial or complete cure for a disorder and/or adverse effect attributable to the disorder or infection.

The term “bioavailability” refers to the fraction of an administered dose of unchanged drug that reaches the systemic circulation. For example, when a medication is administered intravenously, its bioavailability is 100%. However, when a medication is administered via other routes (such as orally), its bioavailability generally decreases due to incomplete absorption and first-pass metabolism. Bioavailability is one of the essential tools in pharmacokinetics, as bioavailability must be considered when calculating dosages for non-intravenous routes of administration.

The term “erythritol” refers to a four-carbon sugar alcohol that is often used as a food additive and sugar substitute. It is naturally occurring and can be made from corn using enzymes and fermentation. Its formula is C₄H₁₀O₄, or HO(CH₂)(CHOH)₂(CH₂)OH.

The term Cutibacterium acnes (formerly Propionibacterium acnes) or “C. acnes” refers to a relatively slow growing, typically aerotolerant anaerobic, gram-positive bacterium (rod) linked to the skin condition of acne. It can also cause chronic blepharitis and endophthalmitis, the latter particularly following intraocular surgery. Its genome has been sequenced and a study has shown several genes can generate enzymes for degrading skin and proteins that may be immunogenic (activating the immune system). The species is largely commensal and part of the skin flora present on most healthy adult humans' skin. It is usually barely detectable on the skin of healthy preadolescents. It lives, among other things, primarily on fatty acids in sebum secreted by sebaceous glands in the follicles. It may also be found throughout the gastrointestinal tract.

The term “microcomedone” refers to clinically non-visible central precursor lesions of acne that are induced by sebaceous hyperplasia as well as altered follicular growth and differentiation and evolve into both comedones and inflammatory lesions. Targeting microcomedone formation can be effective in the prevention and therapeutic control of acne. Every comedone and inflamed pimple begins its life as a microcomedone.

The term “medical device” refers generally to any device intended to be used for medical purposes. Certain medical devices (e.g., those used invasively or at/near a body orifice) present risks of infection. For example, intravenous drips, urinary catheters and wound drains, are often associated with infections. Invasive devices including endotracheal tubes (ETT), urinary catheters (UC), and central venous catheters (CVC) are associated with increased infection rates in intensive care unit (ICU) settings.

Accordingly, a medical device can be an intravenous (IV) catheter, a central line catheter (e.g., PICC, CVC or port catheter) or a component used for infusion of a medicament (e.g., a chemotherapeutic agent).

The term “invasive medical device” refers to one that is intended by the manufacturer to be used, in whole or in part, inside the body of a human. Foreign body-related infections (FBRIs) can accumulate on the surfaces of medical devices and proliferate, particularly in catheter-related infections. Examples of invasive medical devices (for short term use) include: clamps, infusion cannulae, skin closure devices or temporary filling materials, some surgical retractors for example, chest retractors for cardiac surgery, tissue adhesives, tissue expanders and haemostatic sponges. Examples of invasive medical devices (for long term use) include: implantable joint replacements, shunts, stents, nails, plates and screws, intra-ocular lenses, infusion ports, peripheral vascular grafts, bone cements, maxillo-facial implants, prosthetic heart valves, aneurysm clips, vascular prostheses, spinal stents, vascular stents, CNS electrodes, cardiovascular sutures, absorbable sutures, bioactive adhesives and implants through the attachment of surface coatings such as phosphorylcholine.

Rechargeable non-active drug delivery systems. Invasive medical devices can also include medical implants including: breast implants, cerebral spinal fluid (CSF) shunt systems, cochlear implants, essure permanent birth control, hernia surgical mesh implants, Metal-on-Metal hip implants, phakic intraocular lenses, urogynecologic surgical mesh implants, pacemakers, contraceptive intrauterine devices, intraocular lenses and hip implants.

The term “catheter” refers to a tubular-shaped instrument, usually flexible, which is designed to be passed through a body channel for withdrawing fluids from or inserting fluids into a body cavity. A catheter can be a “cannula,” which is a tube for insertion into a vessel, duct or cavity, usually for draining of fluids or for administration of medical fluids such as oxygen gas, liquid medication, etc. A “urinary catheter,” refers to a catheter for insertion into the urethra to collect urine from the urinary bladder. Long-term catheterization is considered when other methods are not effective or practical, as long-term use can result in bacteriuria, UTI, blockage and bypassing (leakage around the catheter). In particular, the two main indications for long-term indwelling catheters are urinary retention and urinary incontinence.

The terms “coat” or “coating” (verb form) refer to the act of covering a surface with a composition for a sufficient period of time to impregnate the surface with the composition. When the composition is a solution of solute components in a solvent, coating may further involve drying the composition such that at least a portion of the solvent in the composition on the surface is removed and solute components remain on the surface.

The terms “coat” or “coating” (noun form) refer to a layer of material that covers a surface. In reference to a medical device, the entire surface or part of the surface may have a coating. The coat (noun) may include erythritol, zinc and other agents that remain on a surface of a device after drying.

The term “coated” (in adjective form) as used herein refers to a surface having a coat or coating.

The terms “coating solution,” or “coating composition” are used interchangeably herein and refer to a solution or suspension containing a combination of erythritol, zinc and other agents (active and inert) useful to coat a surface of a medical device.

The term “feminine hygiene products” refers generally to personal care products used during menstruation, vaginal discharge, and other bodily functions related to the vulva and vagina. Products that are used during menstruation may also be called menstrual hygiene products, including menstrual pads, tampons, pantyliners, menstrual cups, menstrual sponges and period panties. Feminine hygiene products also include products meant to cleanse the vulva or vagina, such as douches, feminine wipes, and soap.

The term “toxic shock syndrome” refers to a rare, life-threatening complication of certain types of bacterial infections. Conventional efforts to help prevent toxic shock syndrome include using the lowest needed absorbency of tampon and changing tampons at least every four to eight hours.

The term “wet wipe,” “moist towelette,” “disposable wipe,” “disinfecting wipe” or a “baby wipe” refers to a small to medium-sized moistened piece of plastic or cloth that either comes folded and individually wrapped for convenience or, in the case of dispensers, as a large roll with individual wipes that can be torn off. Wet wipes are used for cleaning purposes like personal hygiene and household cleaning; each is a separate product depending on the chemicals added and medical or office cleaning wipes are not intended for skin hygiene.

The term “Irritant diaper dermatitis” or “diaper rash” refers to skin rash in the diaper area that are caused by various skin disorders and/or irritants. Generic irritant diaper dermatitis is characterized by joined patches of erythema and scaling mainly seen on the convex surfaces, with the skin folds spared. Diaper dermatitis with secondary bacterial or fungal involvement tends to spread to concave surfaces (i.e. skin folds), as well as convex surfaces, and often exhibits a central red, beefy erythema with satellite pustules around the border. It is usually considered a form of irritant contact dermatitis. The word “diaper” is in the name not because the diaper itself causes the rash but rather because the rash is associated with diaper use, being caused by the materials trapped by the diaper (usually feces). Allergic contact dermatitis has also been suggested, but there is little evidence for this cause. In adults with incontinence (fecal, urinary, or both), the rash is sometimes called incontinence-associated dermatitis (IAD).

The term “diaper candidiasis” is used when a fungal origin is identified. The distinction is important because the treatment is different.

Conventional methods of preventing or treating diaper rash include barrier creams, mild topical cortisones, and antifungal agents. A variety of other inflammatory and infectious processes can occur in the diaper area and an awareness of these secondary types of diaper dermatitis aids in the accurate diagnosis and treatment.

The term “body odor” refers to an odor/smell that is present in all animals and its intensity can be influenced by many factors (e.g., behavioral patterns, survival strategies). In humans, the formation of body odors is caused by factors such as diet, sex, health, and medication, but the major contribution comes from bacterial activity on skin gland secretions. The medical term for body odor is bromhidrosis, which is also known as osmidrosis or ozochrotia. Bromhidrosis is a common condition in people who have gone through puberty, and is usually diagnosed when it negatively affects someone's quality of life, social interactions or self-esteem.

Bromhidrosis can be classified as eccrine or apocrine, exogenous or endogenous. Eccrine bromhidrosis refers to odor from eccrine glands that are distributed over most of the skin surface. Eccrine sweat (important in thermoregulation) is secreted continuously and is normally odorless. Apocrine bromhidrosis refers to odor from apocrine glands (located in the axillae, anogenital region, and breasts) and become active in puberty. Apocrine sweat is secreted intermittently and is normally odorless and is controlled by the sympathetic nervous system. Exogenous body odor refers to skin microbiota (e.g., Corynebacterium species, Staphylococcus hominis, S epidermidis, Cutibacterium avidum and Acinetobacter schindleri). It can also refer to skin infections (e.g., erythrasma, pitted keratolysis, trichomycosis and sphingomonas paucimobilis) and odor from a nasal foreign body. The last category of body odor is endogenous and can result from foods (e.g., garlic, onion, asparagus, curry and alcohol), medications and toxins (e.g., penicillin, bromides, dupilumab), systemic and skin disorders (e.g., obesity, diabetes mellitus, liver disease, renal failure, skin disorders of palms/soles and flexures) and metabolic disorders (e.g., trimethylaminuria, phenylketonuria and hypermethioninaemia).

The term “deodorant” refers to a substance that is applied to the body to prevent or mask body odor caused by bacterial breakdown of perspiration, for example in the armpits, groin, or feet. A subclass of deodorants, called antiperspirants, prevents sweating itself, typically by blocking sweat glands. Deodorants are typically aerosol sprays, creams or roll-on forms.

The term “infection-resistant,” refers to a device (e.g., a coated article) with the ability to reduce or retard adherence, growth or numbers of microorganisms per surface area compared to an uncoated (control) surface. Preferably, bacteria are reduced by at least 20%, at least 50%, at least 75% or at least 80%, and most preferably by at least 90%, at least 95%, at least 98% or at least 99% compared to control, in and/or on the “infection-resistant” item.

The term “lubricious,” refers to a composition or a substance that reduces the friction force of a surface treated, impregnated or coated therewith. A lubricious composition or substance typically possesses a smooth and slippery quality. The term “lubricity” refers to the property or state of being lubricious. Common lubricious agents include glycerin and silicone-based gels. Others include hydrophilic polymers such as polyvinyl pyrrolidone (PVP).

The term “antimicrobial agent” refers to antibiotics, antiseptics, disinfectants and other synthetic moieties, and combinations thereof, that are soluble in organic solvents such as alcohols, ketones, ethers, aldehydes, acetonitrile, acetic acid, formic acid, methylene chloride and chloroform.

In aspects, a formulation or coating solution described herein includes an antibiotic. Classes of antibiotics that can be used include tetracyclines (i.e., minocycline), rifamycins (i.e., rifampin), macrolides (i.e., erythromycin), penicillins (i.e., nafcillin), cephalosporins (i.e., cefazolin), other beta-lactam antibiotics (i.e., imipenem, aztreonam), aminoglycosides (i.e., gentamicin), chloramphenicol, sufonamides (i.e., sulfamethoxazole), glycopeptides (i.e., vancomycin), quinolones (i.e., ciprofloxacin), fusidic acid, trimethoprim, metronidazole, clindamycin, mupirocin, polyenes (i.e., amphotericin B), azoles (i.e., fluconazole) and beta-lactam inhibitors (i.e., sulbactam). Examples of specific antibiotics that can be used include minocycline, rifampin, erythromycin, nafcillin, cefazolin, imipenem, aztreonam, gentamicin, sulfamethoxazole, vancomycin, ciprofloxacin, trimethoprim, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, clinafloxacin, sulbactam, clavulanic acid, amphotericin B, fluconazole, itraconazole, ketoconazole, and nystatin. Other examples of antibiotics, such as those listed in Sakamoto et al., U.S. Pat. No. 4,642,104, are known in the art and can also be used.

In aspects, a formulation or coating solution described herein includes an antiseptic and/or disinfectant. Examples of antiseptics and disinfectants are benzethonium chloride, thymol, a-terpineol, methylisothiazolone, cetylpyridinium, chloroxylenol, hexachlorophene, cationic biguanides (i.e., chlorhexidine, cyclohexidine), methylene chloride, iodine and iodophores (i.e., povidone-iodine), triclosan, furan medical preparations (i.e., nitrofurantoin, nitrofurazone), methenamine, aldehydes (glutaraldehyde, formaldehyde) and alcohols. Other examples of antiseptics and disinfectants will readily suggest themselves to those of ordinary skill in the art.

The term “compound” or “formulation” refers to a combined mixture of multiple ingredients. A compound or formulation may further refer to a customized formulation of ingredients designed for therapeutic, prophylactic, preventative and aseptic purposes.

The term “an effective amount” refers to the amount of the defined component sufficient to achieve the desired chemical composition or the desired therapeutic result. The desired result is the alleviation or amelioration of the signs, symptoms, or causes of a skin disease, or any other desired alteration of a biological system including stopping the propagation of biofilm. For treating acne, the signs and symptoms can include the presence of a number of comedones, papules and/or pustules. When the desired result is a therapeutic response, the effective amount will, without limitation, vary depending upon the specific disease or symptom to be treated or alleviated, the age, gender, sex and weight of the subject to be treated, the dosing regimen of the formulation, the severity of the disease condition, the manner of administration and the like, all of which can be determined readily by one of skill in the art. A desired effect may, without necessarily being therapeutic, also be a cosmetic effect, in particular for treatment for disorders of the skin described herein. A desired effect may, without necessarily being therapeutic, also be a preventative effect, in particular for preventing the spread of biofilm on living and non-living surfaces.

The terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder. The effect may be preventative in terms of thwarting the spread of biofilm on surfaces, preventing the formation of biofilm, and in some cases eliminating the existence of biofilm on a surface.

The term “treat” in the context of a substrate or device refers to a process of applying a substance to a surface of a material to improve it (e.g., make it resistant to bacterial growth). Treatment can include rinsing a surface or otherwise exposing (e.g., for a longer period of time) the surface to an antimicrobial formulation. The surface can be dried following the treatment step and a portion of the active agent (i.e., erythritol and zinc) can remain on the surface.

An “effective concentration” refers to a sufficient amount of the antimicrobial agent that is added to decrease, prevent or inhibit the growth of bacteria. The amount will vary for each compound and upon known factors such as pharmaceutical characteristics; the type of medical device; age, sex, health and weight of the recipient; and the use and length of use. It is within the skilled artisan's ability to relatively easily determine an effective concentration for each compound.

The term “exfoliation” refers to the removal of dead skin cells and built-up dirt from the surface of the skin. Exfoliation can help skin look brighter and enhance how well your topical skin care products work by improving absorption. Exfoliation can be either physical or chemical. Physical exfoliation includes a process that involves scrubbing or rubbing using a device (e.g., grainy scrubs, dry brushes, netted bath mitts, loofahs, etc.) to physically disrupt the surface of the skin. Chemical peels involve applying a dilute acid (or enzyme) onto the skin for a short period of time to dissolve away dead skin. Chemical exfoliants include alpha-hydroxy acids (AHAs), beta-hydroxy acids (BHAs), retinol and some enzymes.

The term “dermabrasion” refers to a skin-resurfacing procedure that uses a rapidly rotating device to sand the outer layers of skin. Immediately after dermabrasion, treated skin will be reddish and swollen. The skin that grows back is usually smoother. Dermabrasion is a more aggressive technique than microdermabrasion, and it erases significantly more wrinkles or scars. Therefore, dermabrasion often causes bleeding, while microdermabrasion does not. Dermabrasion requires time to recover (i.e., downtime) while microdermabrasion resolves without any downtime.

The term “microdermabrasion” refers to a method of using a mildly abrasive substance or instrument to gently sand the skin which removes the outer layer. A common method involves mechanical rubbing by a handpiece that emits a stream of sand or grit. For example, a handpiece can be used to direct an air flow containing tiny crystals of aluminum oxide, sodium chloride, or sodium bicarbonate. The momentum of the grit tends to wear away two to three cell layers of the skin with each pass of the handpiece. Alternative methods (e.g., “crystal-free” microdermabrasion) use a diamond-tipped handpiece without a stream of grit.

The term “hydradermabrasion” or “HydraFacial®” refers to a method that combines microdermabrasion with administration of a skin formulation (e.g., moisturizer, rejuvenating serum, etc.). The tip of a machine (HydraFacial® tip) clears out pores on the surface of the skin and simultaneously delivers topical moisturizers. The method can be described in three steps: (a) cleanse and peel, (b) extract and hydrate and (c) infuse and protect. Other systems/methods with similar approaches include DiamondGlow® and SkinMedica®.

The term “cosmeceutical” refers to a substance that includes a cosmetic with one or more “biologically active ingredients.” Products which are similar in perceived benefits but ingested orally are known as nutricosmetics.

The term “chemical permeation enhancer” or “CPE” refers to a molecule that interacts with the constituents of skin's outermost layer, the stratum corneum (SC), and increases its permeability.

The term “dermal filler” (also referred to as injectable implants, soft tissue fillers, lip and facial fillers, or wrinkle fillers) are medical device implants for use in helping to create a smoother and/or fuller appearance in the face, including nasolabial folds. Dermal fillers can help diminish facial lines and restore volume and fullness in the face. Common dermal fillers include: Hyaluronic acid (Restylane®), calcium hydroxylapatite (Radiesse®), poly-L-lactic acid (Sculptra®) and polymethylmethacrylate (PMMA) (Bellafill®).

The term “granuloma” refers to a growth that forms when immune cells clump together and create tiny nodules at the site of the infection or inflammation. A granuloma is the body's way to contain an area of bacterial, viral or fungal infection so it can try to keep it from spreading or to isolate irritants or foreign objects.

The term “folliculitis” refers to a common skin condition that can be described as inflammation of the hair follicle. It typically appears as small bumps scattered throughout the scalp and can appear similar to acne. The bumps can sometimes feel sore. On subject with darker skin tones, the bumps can be a dark shade of red or light purple around the periphery.

The term “impetigo” refers to a common skin infection that often affects children. It is a contagious bacterial infection. Staphylococcus bacteria live on the skin and are mostly harmless, but they can cause an infection if they enter damaged skin. Another bacterium called Streptococcus can also cause impetigo. This bacteria can spread from person to person by skin contact, touching objects, or sneezing and coughing. Impetigo most commonly affects the face, particularly the area around the nose and mouth, but it can affect any part of the body where the skin is broken. This includes the scalp. Impetigo can also spread from the original site to other areas of the body.

The term “suppository” refers to a solid medical preparation in a roughly conical or cylindrical shape, designed to be inserted into the rectum or vagina to dissolve.

The term “skin appearance” refers to the outward characteristic features that are visible to others. Improvements in skin appearance can include, for example, reduced visibility of fine lines, wrinkles, and other forms of uneven or rough surface texture associated with aged or photo damaged skin. Many different research fields have conducted extensive research on the appearance of skin. The fields of medicine, cosmetology, computer graphics, and computer vision have been most active in the study of skin appearance. Studies in each of these fields has provided knowledge and insights regarding different aspects of skin appearance.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13^{th} Ed), Mack Publishing Company, Easton, PA—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

As the patients and subjects of the invention method are, in addition to humans, veterinary subjects, formulations suitable for these subjects are also appropriate. Such subjects include livestock and pets as well as sports animals such as horses, greyhounds, and the like.

### DETAILED DESCRIPTION

Embodiments include formulations and methods for treating a skin condition such as acne. Conventional methods of treating acne include topical antibiotics. Commonly used antibiotics, either applied to the skin or taken orally, include clindamycin, erythromycin, metronidazole, sulfacetamide, and tetracyclines. However, topical formulations are often ineffective in part because the active agents remain at the surface of the skin. Oral antibiotics are generally over-prescribed and can lead to antibiotic-resistant C. acnes strains.

Small amounts of zinc are essential for metabolic processes and have a positive impact on bone formation. It has been proposed that zinc ions act as an antimicrobial agent by deactivating proteins, causing structural changes in microbial membranes and affecting microbial nucleic acids, although the efficacy is insufficient to eradicate mature biofilms. Sugar alcohols such as xylitol also have antimicrobial effects. Xylitol is often used to control oral biofilms due to its safety and ability to inhibit the formation of biofilms. In particular, xylitol-containing chewing gums are widely used worldwide.

The combination of zinc chloride-erythritol can have an additive or synergistic effect in fighting bacteria. Zinc and sugar alcohols have demonstrated antimicrobial activity and can be used for removing biofilms.

Without being bound by theory, Applicant proposes that the compounds described herein are effective treatments for acne due, at least in part, to the antimicrobial effect of the zinc chloride-erythritol mixture. Applicants propose that acne can be effectively treated by preventing or ameliorating bacterial growth at or near skin pores and hair follicles. The topical formulation allows the zinc chloride-erythritol mixture to effectively reach regions at or below the outer layer of skin (i.e., the epidermis). Accordingly, embodiments include formulations and methods for topical administration of erythritol and zinc.

In one embodiment, the erythritol and zinc are used at a molar ratio of about 3:1. Data suggests that this ratio is most effective against bacteria, including Cutibacterium acnes (i.e., C. acnes). The erythritol and zinc can be combined in a topical lotion. The lotion can also have a humectant, an emulsifier and an emollient.

In another embodiment, the zinc and erythritol can be combined with one or more additional acne medications. For example, the formulation can be co-administered with benzoyl peroxide, a retinoid, a steroid, an antibiotic, azelaic acid, salicylic acid, dapsone, a contraceptive, an anti-androgen agent or isotretinoin to the subject. In one embodiment, the agent works synergistically with zinc and erythritol. In one embodiment, the second agent reduces inflammation. In another embodiment, the second agent is an exfoliant. For example, topical retinoids can work in conjunction with topical antibiotics. They can exfoliate the skin and reduce the formation of comedones (blocked pores).

As described herein, zinc chloride and erythritol can be used at a specific molar ratio to one another. In one embodiment, a mixture of zinc chloride and erythritol has a molar ratio of about 1:3. In one embodiment, a mixture of zinc chloride and erythritol has a molar ratio of about 1:1.3. In one embodiment, a mixture of zinc chloride and erythritol has a molar ratio of about 1:1.5. In one embodiment, a mixture of zinc chloride and erythritol has a molar ratio of about 1:1.75. In one embodiment, a mixture of zinc chloride and erythritol has a molar ratio of about 1:2. In one embodiment, a mixture of zinc chloride and erythritol has a molar ratio of about 1:4. In one embodiment, a mixture of zinc chloride and erythritol has a molar ratio of about 1:5. In one embodiment, a mixture of zinc chloride and erythritol has a molar ratio of about 1:6. In one embodiment, zinc chloride and erythritol are used in equimolar amounts.

### Topical Formulation Components

Embodiments include a lotion or cream for administration of medicaments to a subject. It is placed on the skin to deliver a specific dose of an agent through the skin. The agent can be delivered across the skin into a localized subdermal location (e.g., near areas with acne). For example, a lotion can inhibit growth of C. acnes and reduce inflammation. The lotion or cream can be applied directly to the affected area such as the face. In an embodiment, a topical formulation is applied to the skin and the mucous membranes of the eye (an eye ointment), chest, vulva, anus, and nose.

In an embodiment, a topical formulation can be in the form of any of the following: a liquid, lotion, crème, serum, spray, ointment, gel, foam, liquid foundation or balm. A solution can be one or more of a water or alcoholic lotion containing dissolved active ingredients.

In another embodiment, a topical formulation can be a lotion, which is generally thicker than a solution, and in some embodiments, it can include an oil as well as water or an alcohol. In an embodiment, a lotion can separate into two or more different parts with time such that the lotion may need to be shaken into suspension before use.

In a further embodiment, a topical formulation can be a cream. A cream can be thicker than a lotion. One result is that a cream is capable of maintaining its shape. In an embodiment, a lotion includes a 50:50 emulsion of oil and water. A cream can include a preservative to extend its shelf life.

In another embodiment, a topical formulation can be in the form of an ointment. In an embodiment, an ointment includes a composition that in an embodiment is a semi-solid, water-free or nearly water-free (e.g., 80% oil). An ointment can be greasy, sticky, emollient, protective and/or occlusive. An ointment can be homogeneous, viscous, semi-solid preparation, which in some embodiments are greasy, a thick oil (oil 80%-water 20%) with a high viscosity, that is intended for external application to the skin or mucous membranes. Ointments do not always require the addition of a preservative, so ointments are less likely to result in a contact allergy. An ointment generally includes one or more of a hydrocarbon (paraffin), wool fat, beeswax, macrogols, emulsifying wax, cetrimide and/or a vegetable oil (olive oil, arachis oil, coconut oil).

In another embodiment, a topical formulation can be a gel. In an embodiment, a gel includes an aqueous and/or alcoholic monophasic semisolid emulsion and/or a cellulose. A gel can, in an embodiment, liquefy upon contact with skin. Gels often includes preservatives and fragrances. In an embodiment, a gel can include a cellulose cut with alcohol or acetone.

In a further embodiment, a topical formulation can be a paste. In an embodiment, a paste includes of a concentrated suspension of oil, water and/or powder.

In an embodiment, a topical formulation can be an aerosol, foam or spray. Generally, an aerosol, foam or spray is comprised of a solution with a pressurized propellant. In a further embodiment, a topical formulation is a powder.

In an embodiment, a medical device coating can be a foam. In other embodiments, the medical device coating can be a gel. In yet other embodiments, the medical device coating can be a paste. In other embodiments, a medical device coating can be a powder. In yet other embodiments, a medical device coating may be an aerosol or a spray.

A powder can include, for example, a talc (a mineral) or a starch (corn starch, corn cob powder or other vegetable starch). A powder can be inhaled, for example, for a nasal surgery.

In an embodiment, a topical formulation is a solid. A solid can include an antiperspirant or a sunscreen stick, which may melt on reaching body temperature (e.g., a suppository).

In a further embodiment, a topical formulation is a tincture. In an embodiment, a tincture includes a high percentage of alcohol.

The medical device coating of the present invention may be stowed or packaged in myriad receptacles and reservoirs. The medical device coating may be provided in a vial, a blister pack, a container, a syringe, a carpule, a cartridge, a tablet, a capsule, or may be pre-applied to any suitable surface of a medical device prior to packaging.

In an embodiment, a topical formulation includes a vesicle within which the active agents, e.g., erythritol and zinc, are encapsulated and then released at a later time. The release can occur following application to the site on the skin or the release can occur over a period of time to ensure. The vesicle can include a liposome or a nanoparticle. The nanoparticle can include a lipid-based nanoparticles, niosomes, transfersomes, ethosomes, dendrimers, micellar nanoparticles, polymeric as well as metallic and magnetic nanostructures. A nanoparticle can be of any size, but preferably less than 100 nm, less than 95 nm, less than 90 nm, less than 85 nm, less than 80 nm, less than 75 nm, less than 70 nm, less than 65 nm, less than 60 nm, less than 55 nm, less than 50 nm, less than 45 nm, less than 40 nm, less than 35 nm, less than 30 nm, less than 25 nm, less than 20 nm, less than 15 nm, less than 10 nm, or less than 5 nm in diameter.

In an embodiment, a topical formulation is administered using a pressure-driven jet. The threshold velocity for penetration into human skin in an embodiment is 100-200 m/s. In another embodiment, the threshold velocity is at least 100 m/s, at least 110 m/s, at least 120 m/s, at least 130 m/s, at least 140 m/s, at least 150 m/s, at least 160 m/s, at least 170 m/s, at least 180 m/s, at least 190 m/s, at least 200 m/s. In another embodiment, the threshold velocity is no more than 100 m/s, no more than 110 m/s, no more than 120 m/s, no more than 130 m/s, no more than 140 m/s, no more than 150 m/s, no more than 160 m/s, no more than 170 m/s, no more than 180 m/s, no more than 190 m/s, no more than 200 m/s.

In another embodiment, a topical or transdermal formulation includes a transdermal patch. An advantage of a transdermal patch is that it provides precise dosing of an active agent (i.e., a sugar alcohol and/or zinc). A transdermal patch can include an adhesive to allow for fixation of the patch on the body of a patient. It can also include a liner that protects the patch during storage. For a transdermal patch that includes a liner, the liner can be removed prior to use. A transdermal patch can also include the active ingredient, including, but limited to a drug in solution in direct contact with a liner and becoming exposed upon removal of the liner. The active ingredient can also be contained in a reservoir. The active ingredient can be part of a formulation that comprises a permeation enhancer to promote the increase in the delivery of the drug transdermally. The transdermal patch can also include an adhesive that serves to adhere the components of the patch together along with adhering the patch to the skin. A transdermal patch can also include a membrane that in an embodiment, is capable of controlling the release of the drug from the reservoir and/or different layer of the patch. A transdermal patch can also include a backing that protects the patch from the outer environment. A transdermal patch can also include a matrix filler that provides bulk to the matrix and/or a stiffening agent. A transdermal patch can also include other components, including a stabilizer (e.g., an antioxidant) or a preservative. A transdermal patch can include a single-layer or a multi-layer active agent. A transdermal patch can also include a vapor patch that serves to adhere the various layers together but also releases a vapor. The vapor can include an essential oil.

In an embodiment, a topical formulation is administered through the use of a sponge as a carrier for a liquid medicine.

In another embodiment, a topical formulation is administered through a tape. In an embodiment, a tape can be a cordran tape.

In one embodiment, a topical formulation comprises an aluminum acetate topical solution. An aluminum acetate topical solution is generally colorless, with a faint acetous odor and sweetish taste. It is applied topically as an astringent after dilution with 10-40 parts of water. An aluminum acetate topical formulation is used in many types of dermatologic creams, lotions, and pastes. An aluminum acetate topical formulation can be premeasured and packed as tablets and powders.

An advantage of a transdermal drug delivery route over other types of delivery is that the formulation can provide a controlled release of the agent. Further, transdermal administration is not affected by stomach or digestive issues. Oral consumption of erythritol and zinc would not be expected to significantly help treat acne, even in high doses. Further, people can benefit from drugs that are absorbed slowly and regularly. With a transdermal formulation, a medicament can be released in small quantities over a long period of time.

Other advantages are related to dosing. Large doses of agents can cause dose-dependent toxicity in many cases. For example, oral administration of vitamin A can result in hypervitaminosis A. The main problems associated with the vitamin A are its half-life, fast absorption (due to lipophilicity) and its toxicity (due to high loading and frequent dosing). Also, some drugs undergo first-pass metabolism, which prevents their delivery to the desired site of action. Furthermore, many hydrophilic or lipophilic drugs show either poor dissolution or poor absorption on oral administration. With a transdermal formulation, the effective concentration of an agent can be applied at the desired site without painful delivery.

Administration in Conjunction with Exfoliation

The skin care compositions described herein are generally applied topically and may take the form of a liquid, lotion, crème, serum, spray, ointment, gel, foam, liquid foundation or balm and may be presented as a cosmetic or make-up product, antiperspirant, or another topically applicable health and beauty aid and/or pharmacological product. These types and forms of skin care compositions may themselves be in the form of emulsions, dispersions, liposomes, coacervates and the like. The skin care compositions may also take the form of various articles such as pads, swabs, wipes, sponges, and the like that are saturated with or otherwise contain or hold the actual skin care composition but which release the same or leave a film of the same when swiped across the skin surface. The compositions can be applied subsequent to and/or simultaneously with exfoliation.

Microdermabrasion is used clinically for cosmetic and therapeutic purposes. Such techniques deliver mild abrasives such as aluminum oxide crystals, vitamin C crystals, and other types of mild abrasives to remove the top layer of the skin which is mostly made up of dead skin cells. This process promotes the production of new cells in the basal layer of the dermis. Modern microdermabrasion cosmetic therapies and procedures have been successful in treating patients in the appearance of, for example, blotchy skin, small scars, rhytides (wrinkles), keratoses, large pores, milia (non-inflamed whiteheads caused by keratin-clogged pores), or sebaceous hyperplasia, while minimizing detrimental conditions caused by ablative techniques. These therapies and procedures are often administered in a series of treatments (e.g., four to eight) treatments at approximately two-week intervals.

Modern microdermabrasion techniques often use a device that can simultaneously exfoliate, clean debris and deliver a formulation (i.e., boosters or serums) to the surface of the skin. A particular formulation can be used based on the goal of the treatment, which may include reducing the appearance of hyperpigmentation, fine lines and wrinkles, dull skin, or enlarged pores. Aspects of the invention include compounds and methods of using erythritol and zinc (at a molar ratio of about 3:1) in these formulations.

Devices that combine microdermabrasion to exfoliate and cleanse the skin while administering a formulation are known in the art. Similarly, methods that combine microdermabrasion, suction massage and application of serums/moisturizers are described in the art. See, for example, Freedman, B. M. (2008), Hydradermabrasion: an innovative modality for nonablative facial rejuvenation. Journal of Cosmetic Dermatology, 7: 275-280. Also see U.S. Pat. Nos. 7,789,886, 6,629,983, 8,066,716, 6,592,595, 8,337,513, 6,673,082, 7,678,120 and 6,527,783. Hydradermabrasion's mechanism of action can include: (a) mechanical stimulation to activate the basal layer and (b) thickening and smoothing the epidermis. Resulting fibroblast activity can promote extracellular matrix deposition and dermal thickening. Antioxidants introduced through the procedure hydrate can decrease inflammation in the skin and reverse photo damage, while protecting lipid membranes, collagen fibers and enzyme systems. A single device can be configured to deliver microdermabrasion, suction massage and administration of a serum/moisturizer. The device is typically operated by a healthcare professional, though, it is contemplated that similar devices can be configured for personal (i.e., home) use.

As described above, the combination of zinc chloride-erythritol can have an additive or synergistic effect in fighting bacteria. The combination has demonstrated antimicrobial activity and can be used for removing biofilms. Applicants propose that the formulations described herein can be used against bacteria (and to ameliorate its deleterious effects) safely and effectively. The topical formulation allows the zinc chloride-erythritol mixture to effectively reach regions at or below the outer layer of skin (i.e., the epidermis). Accordingly, embodiments include formulations and methods for topical administration of erythritol and zinc in combination with microdermabrasion. In one embodiment, the erythritol and zinc are used at a molar ratio of about 3:1.

In any of the embodiments described and/or illustrated herein, or variations thereof, treatment fluids and/or other materials can be administered via the tip of a microdermabrasion handpiece (i.e., Hydrafacial® tip) assembly using one or more methods. For example, formulations containing serums and other agents can be administered simultaneously with mechanical exfoliation. Such serums, compositions, other fluids or substances can be pre-mixed so that they are delivered to the tip and the skin unmodified or substantially unmodified.

In other embodiments, serums, fluids, gels or other materials can be in the form of a pack or container of dry granular material, viscous gels and/or the like. Such packs can be mixed with water or some other fluid by a user to a desired concentration. In other embodiments, one or more treatment materials can be impregnated or otherwise embedded into the tips of the handpiece assemblies. Thus, such materials (e.g., powders, solids, gels, etc.) can advantageously dissolve when they contact water, saline or some other liquid. In still other embodiments, the treatment materials can be contained within a capsule, tablet or other enclosure. Such enclosures can be configured to dissolve when placed in water or some other fluid. Therefore, in some embodiments, a user may be required to place a capsule, the contents of a pack or some other materials into a canister and add water or other fluid before use.

In some embodiments, one or more serums or other substances can be delivered to the treatment surface of a handpiece assembly to treat a particular skin condition. For example, the system can be used to treat acne, dry or oily skin, fine lines, sun-damaged skin, other skin diseases or disorders and/or the like.

In some embodiments, the serums, other materials and/or a combination of such serums or other materials can be utilized for the treatment of substantially most or all skin types. For example, such serums and/or other materials can be used when the handpiece assembly exfoliates skin.

In aspects, the formulation also includes an emollient, a humectant, an antioxidant and/or an anti-inflammatory ingredient. Suitable emollients include those agents known for softening the skin which may be selected from hydrocarbons, fatty acids, fatty alcohols and esters. Petrolatum is a common hydrocarbon type of emollient conditioning agent. Other hydrocarbons that may be employed include alkyl benzoate, mineral oil, polyolefins such as polydecene, and paraffins, such as isohexadecane. Fatty acids and alcohols typically have from about 10 to 30 carbon atoms. Illustrative are myristic, isostearic, hydroxystearic, oleic, linoleic, ricinoleic, behenic and eruicic acids and alcohols. Oily ester emollients may be those selected from one or more of the following, triglyceride esters, acetoglyceride esters, ethoxylated glycerides, alkyl esters of fatty acids, ether esters, polyhydric alcohol esters and wax esters. Additional emollients or hydrophobic agents include C12 to C15 alkyl benzoate, dioctyladipate, octyl stearate, octyldodecanol, hexyl laurate, octyldodecyl neopentanoate, cyclomethicone, dicapryl ether, dimethicone, phenyl trimethicone, isopropyl myristate, capriylic/capric, triglycerides; propylene glycol dicaprylate/dicaprate and decyl oleate.

Suitable humectants include various polyhydric alcohols, especially polyalkylene glycols and, more preferably, alkylene polyols and their derivatives. Exemplary humectants include propylene glycol, dipropylene glycol, polypropylene glycol, polyethylene glycol, sorbitol, 2-pyrrolidone-5-carboxylate, hydroxypropyl sorbitol, hexylene glycol, xylitol, ethoxydiglycol 1,3-butylene glycol, 1,2,6-hexanetriol, glycerin, ethoxylated glycerin, propoxylated glycerin, compatible solutes, such as ectoin, hydroxectoin, taurines, carnithine, acetyl carnithine and mixtures thereof. When employed in effective amounts, generally from 1 to 30%, preferably from 2 to 20%, by weight of the topical composition, these additives serve as skin moisturizers as well as reduce scaling and stimulate the removal of built-up scale from the skin.

Suitable anti-inflammatory ingredients include, for example, bisabolol, curcurmin and its derivatives, retinoids, flavonoids, meroterpenes (such as Bakuchiol or its derivatives) and other polyphenolics etc. These and other anti-inflammatory agents, as well as additional antioxidants and the like are known in the art (see, e.g., US 2005/0048008A1).

In addition to those benefits described above, it is to be appreciated that use of the compositions described herein, regardless of whether one is manifesting a problem to be addressed or not, provide a number of additional benefits to the skin. For example, use of the inventive compositions (with or without the step of microdermabrasion) may help with thickening the keratinous tissue (i.e., building the epidermis and/or dermis layers of the skin), thereby preventing and/or retarding atrophy of human skin; preventing and/or retarding the appearance of spider veins and/or red blotchiness on human skin; preventing and/or retarding the appearance of dark circles under the eye; preventing and/or retarding sallowness and/or sagging of human skin; soften and/or smooth lips; preventing and/or relieving itch of human skin, regulating skin texture (e.g., wrinkles and fine lines), improving skin color (e.g., redness, freckles), etc. In essence, the long-term benefits of the use of the methods and compositions described herein include the lessening or delayed manifestation, possibly even the prevention or repair, of skin damage owing to the natural process of skin aging as well as skin damage due to environmental factors, especially sun exposure. Generally, the use of these methods and compositions will manifest itself in an overall improved skin quality as compared to skin which has not been treated.

### Antimicrobial Coating

The colonization of bacteria on the surfaces of medical devices, particularly implanted devices, presents a serious risk to patients. A biofilm may form on surfaces of a device which can harbor microorganisms and encourage further adherence of bacteria. The formation of biofilms on the surface of medical devices can be detrimental to the integrity of the medical device, present health risks and inhibit flow through the lumens of medical devices.

Although coating or cleaning medical devices with antimicrobial agents, such as antibiotics or antiseptics, can be effective in killing or inhibiting growth of free-floating or “planktonic” organisms not adhered to the device surface, such antimicrobial agents are generally much less active against the microorganisms that are deeply embedded within the biofilm. Conventional antimicrobial agents may be unable to penetrate the biofilm. The failure of the antimicrobial agents to sufficiently remove the microorganisms is therefore largely due to the protective effect of the biofilm which prevents diffusion of antimicrobial deep into the biofilm layer to eliminate the microorganisms proliferating therein.

Embodiments of the invention are intended to assist in maintaining an environment on and around the medical devices that retards or reduces bacteria (and biofilm) by coating medical device with a composition that imparts anti-bacterial properties. The formulations described herein can also be used as a “wash” or “rinse” to remove biofilm from a device.

While some catheters or other medical devices have been coated with antimicrobial coatings to reduce bacterial growth, conventional methods are generally complex and have not demonstrated long-term clinical benefit. For example, one method of coating the devices would be to first apply or absorb to the surface of the medical device a layer of surfactant, such as tridodecylmethyl ammonium chloride (TDMAC), followed by an antibiotic coating layer. Another method involves first coating a surface with benzalkonium chloride followed by ionic bonding of an antibiotic composition. See, e.g., Solomon, D. D. and Sherertz, R. J., J. Controlled Release, 6:343-352 (1987) and U.S. Pat. No. 4,442,133. Other methods of coating surfaces of medical devices with antibiotics are taught in U.S. Pat. Nos. 4,895,566; 4,917,686; 4,107,121; 5,013,306; and 4,952,419. These and similar methods of coating medical devices with antimicrobial agents (antibiotics and/or antiseptics) appear in numerous patents and publications. However, conventional coating methods have generally been ineffective because they are complicated and the coating diminishes soon after application.

The use of silver compounds in antimicrobial coatings for medical devices is also known in the art. The antiseptic activity of silver compounds is a known property that has been utilized for many years in topical formulations. Silver can be used topically either as a metal or as silver salts. Bactericidal amounts of silver ions (Ag+) are released from coating. A specific advantage of silver ion as an antibacterial agent is the inability of bacteria to acquire tolerance to it. However, silver compounds (e.g., silver iodide and silver sulfide) have low solubility and are poorly ionized, thus have minimal antibacterial effects at useful concentrations.

The Applicant has discovered that erythritol and zinc can be used effectively as an antimicrobial agent on a surface of a substrate. The antimicrobial coating as described herein can be applied to any and all surfaces that have the potential to host a biofilm. In an embodiment, the antimicrobial coating includes zinc chloride and erythritol at a 1:3 molar ratio that is applied to the surface of a medical device before the medical device is used on a patient. The compound of zinc chloride and erythritol can prevent (and clears or disinfects) the formation of biofilm on a medical device.

Accordingly, embodiments include methods of treating substrate surfaces (e.g., medical devices) to negate and/or reduce the propagation of biofilm. Treatment can include rinsing a surface or otherwise exposing (e.g., for a longer period of time) the surface to an antimicrobial formulation. After the rinsing step, the surface can be dried and a portion of the active agent (i.e., erythritol and zinc) can remain on the surface. In aspects, the antimicrobial formulation includes erythritol and zinc chloride at a 3:1 molar ratio. In aspects, the antimicrobial coating is a solution that includes 6.6 mM zinc chloride and 19.8 mM erythritol. In aspects, the antimicrobial coating is a solution that includes 13.2 mM zinc chloride and 39.6 mM erythritol. In aspects, the antimicrobial coating is a solution that includes 19.8 mM zinc chloride and 59.4 mM erythritol. Higher concentrations (with the same molar ratio) are also contemplated.

In aspects, the antimicrobial coating formulations of the invention are applied to the surface of a substrate material by a spray coat method. The antimicrobial coating formulation can be sprayed on the substrate material surface using standard spraying equipment and methods known in the art. Suitable spraying equipment include sprayers using pressurized air, and sprayers using an ultrasonic spray head, both of which aerosolize the coating solutions.

The coating layer formed on substrate material surface described herein can then be dried by a suitable drying process that includes, for example, air-drying, infrared radiation, convection or radiation drying (e.g., a drying oven), or warm forced air (e.g., heat gun). In the case of multi-layer coatings, the drying step can be performed after formation of each of the inner layers.

The coating formulations of the invention can be applied to a variety of substrate materials, including synthetic and naturally occurring organic and inorganic polymers such as polyethylene, polypropylene, polyacrylates, polycarbonate, polyamides, polyurethane, polyvinylchloride (PVC), polyetherketone (PEEK), polytetrafluroethylene (PTFE), cellulose, silicone and rubber (polyisoprene), plastics, metals, glass, and ceramics. While the coating formulations of the invention may be applied either directly on materials with a hydrophilic surface such as metals, glass and cellulose or optionally on top of a primer undercoat, materials with hydrophobic surfaces such as silicone and PTFE can be subject to a surface pre-treatment step prior to application of the coating.

In aspects, the antimicrobial coating also includes one or more additional antibacterial agents (e.g., a silver compound) or small molecule antibiotic. These include antibiotics such as but not limited to rifampin, gentamicin, vancomycin, neomycin, soframycin, bacitracin, polymycin, synthetic antibiotics including ofloxacin, levofloxacin and ciprofloxacin, antibacterials including biguanides such as chlorhexidine and their salts, alkyl ammonium halides such as benzalkonium chloride cetrimide, domiphen bromide and phenolics such as triclosan.

In aspects, a device is sterilized before and/or after coating a surface of the device. Sterilization is typically accomplished using steam, ethylene oxide (EO) or gamma radiation. NO₂ works as an oxidizer that inactivates microorganisms through degradation of DNA, providing a relatively high sterility assurance level (SAL) at relatively low gas concentrations.

In embodiments, the coating is applied to a critical medical device, a semi-critical medical device or a noncritical medical device. Critical devices include those introduced directly into the blood stream or contact a normally sterile tissue or body space during use. Examples include surgical instruments, implantable devices, irrigation systems for sterile instruments in sterile tissues, endoscopes used in sterile body cavities, and all endoscope biopsy accessories. Semi-critical devices are those that contact intact mucous membranes or non-intact skin, without tissue penetration. Examples include endotracheal tubes, laryngoscope blades and other respiratory equipment, esophageal manometry probes, endo-cavity probes, tonometers and bronchoscopes. Non-critical devices are instruments or medical devices that contact intact skin without tissue penetration. Examples include infusion pumps and ventilators, blood glucose meters, stethoscopes and oximeters.

In embodiments, the coating includes one or more agents to increase lubricity of a medical device such as a catheter. For example, the coating solutions can include lubricity enhancing agents such as, for example, flax seed oil, grape seed oil, avocado oil or other natural oils, silicone oils, or emollient solvents (e.g., Procetyl™ 10 (PPG-10 Cetyl Ether), PPG-3 benzyl ether myristate, ethyl hexyl glycerine, and/or octoxyglycerin). Natural oils also can reduce the inflammation in the urinary tract due to catheterization. Thus, oils and emollients that have useful properties such as lubricity, anti-inflammatory activity, penetration-enhancing activity or other antimicrobial compounds are contemplated for use with certain embodiments of the invention. In a specific embodiment, the lubricity-enhancing agents are included in the coating solution. In this manner, articles for which enhanced lubricity is advantageous, such as catheters, can be treated to impart antimicrobial activity and enhanced lubricity in one treatment or coating step, simplifying manufacture and reducing expense.

Other compounds (e.g., inert components) having other properties also can be added to the coating solutions to improve various properties. For example, it is contemplated that in some embodiments compounds such as the following can be added: preservatives, colorants, dyes, surfactants, antioxidants (e.g., vitamin E, vitamin C), solvents, fillers, pH adjusters, fragrances, and pharmaceuticals (e.g., povidone iodine quaternary ammonium compounds, nitrofurazone and anti-coagulants).

The coatings described herein can be useful for reduction of or prevention of infection by a wide range of gram-positive as well as gram negative bacteria, including, gram positive Staphylococcus aureus, methicillin-resistant S. aureus (MRSA), Staphylococcus epidermidis, Enterococcus faecalis, Staphylococcus saprophyticus, vancomycin-resistant Enterococcus (VRE) spp. and Gram negative Escherichia coli, Klebsiella pneumoniae, Enterobacter spp., Pseudomonas aeruginosa, Proteus mirabilis, Citrobacter spp., and yeast, including Candida albicans.

In embodiments, the coating includes two or more layers, (e.g., bonding and binding coats). Such coatings can include, for example, polyvinylpyrolidone (PVP), polyurethanes, polyacrylic acid (PAA), polyethylene oxide (PEO) and polysaccharide materials. Additional ingredients and/or layers can increase the potency and/or duration of the antimicrobial coating. They can also improve “delayed release” of active agents from the coating.

The antimicrobial agent (i.e., zinc chloride and erythritol) can be present in the coating composition in an amount of from about 0.5% to about 70% of the weight (w/w) of the coating. In other embodiments, the antimicrobial agent is present in the composition in an amount of from about 0.5% to about 30% of the weight of the coating. In certain other embodiments, the antimicrobial agent is present in an amount of from about 0.5% to about 20% of the weight of the coating. Finally, in certain preferred embodiments, the antimicrobial agent is present in an amount of from about 0.5% to about 7.0% of the weight of the coating.

The antimicrobial coating of the present invention provides an effective alternative to caustic agents typically employed when disinfecting medical devices and implements. Further, the formulation remains within a suitable pH range which avoids irritation and further complications typically associated when disinfecting agents are applied to implantable, insertable and/or indwelling medical devices and implements.

The medical coating can also be applied to prostheses to prevent the formation and/or propagation of biofilm. Preventing the formation of biofilm on an implantable or attachable prosthesis can increase chances of biocompatibility of the prosthesis while reducing chances of infection.

In aspects, the antimicrobial formulation includes erythritol and zinc chloride at a 3:1 molar ratio. Other proposed molar ratios of zinc chloride and erythritol may exceed or deceed (fall below the specified threshold or amount) by about 5%, by about 10%, by about 12% by about 15%, by about 20%, by about 25%, by about 30%, by about 35%, by about 40%, by about 45%, by about 50%, by about 55%, by about 60%, by about 65%, by about 70%, by about 75%, by about 80%, by about 85%, by about 90%, by about 95%, by about 100%. Furthermore, it is contemplated that the molar ratio of zinc chloride and erythritol may exceed the aforementioned ratios by about 105%, by about 110%, by about 115%, by about 120%, by about 125%, by about 130%, by about 135%, by about 150%, by about 200%, by about 300%, by about 400%, by about 500%, by about 1000%.

In an embodiment, the molar ratio of erythritol to zinc in the medical device coating is at least 1:1, at least 2:1, at least 3:1, at least 4:1, at least 5:1, at least 6:1, at least 7:1, at least 8:1, at least 9:1, at least 10:1.

In an embodiment, the molar ratio of erythritol to zinc chloride in the medical device coating is about 1:1, about 2:1, about 2.2:1, about 2.5:1, about 3:1, about 3.2:1, about 3.5:1, about 3.7:1, about 4:1, about 5:1, about 6:1, about 7:1, about 8:1, about 9:1, about 10:1.

In an embodiment, the molar ratio of erythritol to zinc in the medical device coating is no more than 1:1, no more than 2:1, no more than 3:1, no more than 4:1, no more than 5:1, no more than 6:1, no more than 7:1, no more than 8:1, no more than 9:1, no more than 10:1.

In an embodiment, the molar ratio of erythritol to zinc chloride in the medical device coating is no more than 1:1, no more than 2:1, no more than 3:1, no more than 4:1, no more than 5:1, no more than 6:1, no more than 7:1, no more than 8:1, no more than 9:1, no more than 10:1.

In other aspects and embodiments, the sugar alcohol in the formulation may be a sugar alcohol other than erythritol such as xylitol or mannitol. Additional sugar alcohols that may be employed to the same, or substantially the same molar ratios as the aforementioned sugar-alcohol constituents include sorbitol, ethylene glycol, glycerol, threitol, arabitol, xylitol, ribitol, mannitol, galactitol, fucitol, iditol, inositol, volemitol, isomalt, maltitol, lactitol, maltotriitol, maltotetraitol and/or polyglycitol.

In embodiments, the coating of the present invention reduces the accumulated surface area of biofilm on a surface by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% or about 100%.

In embodiments, the coating of the present invention retards the pace of biofilm propagation on a surface by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99% or about 100%.

In embodiments, the coating of the present invention lowers/reduced the granuloma rate of a procedure or implant by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99%.

In embodiments, the coating of the present invention retards the pace of biofilm propagation on a surface by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99%.

In embodiments, the coating of the present invention prevents observable formation of biofilm on a surface within about 1 second, within about 2 seconds, within about 3 seconds, within about 4 seconds, within about 5 seconds, within about 7 seconds, within about 10 seconds, within about 15 seconds, within about 20 seconds, within about 25 seconds, within about 30 seconds, within about 35 seconds, within about 40 seconds, within about 45 seconds, within about 50 seconds, within about 55 seconds, within about 60 seconds, within about 1 minute, within about 2 minutes, within about 5 minutes, within about 10 minutes, within about 20 minutes, within about 30 minutes, within about 45 minutes, within about an hour, within about two hours, within about three hours, within about four hours, within about five hours, within about 10 hours, within about 12 hours, within about 14 hours, within about 15 hours, within about 20 hours, within about 22 hours, within about 24 hours, within about 1 day, within about 2 days, within about 3 days, within about 4 days, within about 5 days, within about 6 days, within one week, within about one week, within about 8 days, within about 9 days, within about 10 days, within two weeks, within about two weeks, within three weeks, within about three weeks, within four weeks or later.

In embodiments, the coating is lubricious to ease entry and egress of implantable, insertable and/or indwelling medical devices and implements. The coating can possess an ultra-low viscosity. In aspects, viscosities of the coating may range from 0 to 5 centipoise (cps). More specifically, viscosities of the medical device coating of the present invention may range from 1×10⁻⁵ cps to 5 cps. In other embodiments, the viscosity of a medical device coating may be as low as 1×10⁻⁶ cps. In further embodiments, the viscosity may be as low as 1×10⁻⁷ cps, 1×10⁻⁸ cps, 1×10⁻⁹ cps, or 1×10⁻¹⁰ cps.

Aspects of the present specification disclose that the infection rate associated with medical device implementation (e.g., via surgery, routine medical care, etc.) is reduced by using a coating described herein. The infection rate can be reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity associated with a disease or disorder described herein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Aspects of the present specification disclose the infection rate associated with medical device implementation (e.g., via surgery, routine medical care, etc.) are reduced following application of a medical device coating by about 10% to about 100%, about 20% to about 100%, about 30% to about 100%, about 40% to about 100%, about 50% to about 100%, about 60% to about 100%, about 70% to about 100%, about 80% to about 100%, about 90% to about 100%, about 10% to about 90%, about 20% to about 90%, about 30% to about 90%, about 40% to about 90%, about 50% to about 90%, about 60% to about 90%, about 70% to about 90%, about 80% to about 90%, about 10% to about 80%, about 20% to about 80%, about 30% to about 80%, about 40% to about 80%, about 50% to about 80%, or about 60% to about 80%, about 70% to about 80%, about 10% to about 70%, about 20% to about 70%, about 30% to about 70%, about 40% to about 70%, about 50% to about 70% or about 60% to about 70%.

In aspects, the infection rate associated with medical device implementation (e.g., via surgery, routine medical care, etc.) is reduced by using a coating for the medical device by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the presence of acne is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Aspects of the present specification disclose the infection rate associated with medical device implementation (e.g., via surgery, routine medical care, etc.) is reduced by about 10% to about 100%, about 20% to about 100%, about 30% to about 100%, about 40% to about 100%, about 50% to about 100%, about 60% to about 100%, about 70% to about 100%, about 80% to about 100%, about 90% to about 100%, about 10% to about 90%, about 20% to about 90%, about 30% to about 90%, about 40% to about 90%, about 50% to about 90%, about 60% to about 90%, about 70% to about 90%, about 80% to about 90%, about 10% to about 80%, about 20% to about 80%, about 30% to about 80%, about 40% to about 80%, about 50% to about 80%, or about 60% to about 80%, about 70% to about 80%, about 10% to about 70%, about 20% to about 70%, about 30% to about 70%, about 40% to about 70%, about 50% to about 70% or about 60% to about 70%.

In an embodiment, the period of application of a medical device coating is for 1 second, 2 seconds, 3 seconds, 4 seconds, 5 seconds, 10 seconds, 15 seconds, 20 seconds, 25 seconds, 30 seconds, 35 seconds, 40 seconds, 45 seconds, 50 seconds, 55 seconds, one minute, two minutes, three minutes, four minutes, five minutes, ten minutes, one hour, two hours, three hours, four hours, five hours, six hours, ten hours, 12 hours, 15 hours, 20 hours, 24 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months, 7 months, 8 months, 9 months, 10 months, 11 months, 12 months, or more.

In another embodiment, a medical device coating of the present invention thwarts the growth of biofilm within medical tubing by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a medical device coating disclosed herein thwarts the progression of biofilm within medical tubing by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a medical device coating disclosed herein thwarts the progression of biofilm within medical tubing by, e.g., about 10% to about 100%, about 10% to about 90%, about 10% to about 80%, about 10% to about 70%, about 10% to about 60%, about 10% to about 50%, about 10% to about 40%, about 10% to about 30%, about 10% to about 20%, about 20% to about 100%, about 20% to about 90%, about 20% to about 80%, about 20% to about 70%, about 20% to about 60%, about 20% to about 50%, about 20% to about 40%, about 20% to about 30%, about 30% to about 100%, about 30% to about 90%, about 30% to about 80%, about 30% to about 70%, about 30% to about 60%, about 30% to about 50% or about 30% to about 40%.

In another embodiment, a coating of the present invention thwarts the growth of biofilm on a surface of a medical device, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a coating disclosed herein thwarts the progression of biofilm within a medical device container by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a coating disclosed herein thwarts the progression of biofilm within a medical device container by, e.g., about 10% to about 100%, about 10% to about 90%, about 10% to about 80%, about 10% to about 70%, about 10% to about 60%, about 10% to about 50%, about 10% to about 40%, about 10% to about 30%, about 10% to about 20%, about 20% to about 100%, about 20% to about 90%, about 20% to about 80%, about 20% to about 70%, about 20% to about 60%, about 20% to about 50%, about 20% to about 40%, about 20% to about 30%, about 30% to about 100%, about 30% to about 90%, about 30% to about 80%, about 30% to about 70%, about 30% to about 60%, about 30% to about 50% or about 30% to about 40%.

In an aspect, the concentration of active agent (i.e., erythritol and zinc) for a medical device coating is about 1%, about 1.5%, about 2%, about 3%, about 4%, about 5%, about 7.5%, about 12.5%, about 15%, about 17.5%, about 20%, about 25% or about 30%. In an aspect, the concentration of active agent is at least 0.5%, at least 1%, at least 1.5%, at least 2%, at least 3%, at least 4%, at least 5%, at least 7.5%, at least 12.5%, at least 15%, at least 17.5%, at least 20%, at least 25% or at least 30%. In an aspect, the concentration of active agent is not more than 0.5%, not more than 1%, not more than 1.5%, not more than 2%, not more than 3%, not more than 4%, not more than 5%, not more than 7.5%, not more than 12.5%, not more than 15%, not more than 17.5%, not more than 20%, not more than 25% or not more than 30%.

### Anti-Fruit Fly Coating

Drosophila is a genus of fly, belonging to the family Drosophilidae, whose members are often called “small fruit flies” or pomace flies, vinegar flies, or wine flies, a reference to the characteristic of many species to linger around overripe or rotting fruit. The entire genus of drosophila contains more than 1,500 species and is diverse in appearance, behavior, and breeding habitat.

The Tephritidae are another family that are commonly referred to as fruit flies. The family Tephritidae does not include the biological model organisms of the genus Drosophila (in the family Drosophilidae), which is often called the “common fruit fly”. Nearly 5,000 described species of tephritid fruit fly are categorized in almost 500 genera of the Tephritidae. Description, recategorization, and genetic analyses are constantly changing the taxonomy of this family. To distinguish them from the Drosophilidae, the Tephritidae are sometimes called peacock flies, in reference to their elaborate and colorful markings.

Tephritid fruit flies are among the most destructive agricultural pests of fruits and vegetables worldwide and can impose trade barriers against the movement of fresh tropical commodities. Primary pre-harvest control methods for these flies rely on the spraying of conventional chemical insecticides or bait sprays. However, resistance to these control methods has been reported in fruit flies. Recent studies indicate that sugar alcohols can be used as pesticides and are particularly effective against Tephritidae. Thus, erythritol can be used to control insects, such as tropical tephritid fruit flies.

Accordingly, the formulations and coatings described herein can be used as an alternative to conventional chemical insecticides and/or bait sprays. For example, the coating can be applied to surfaces of produce (e.g., fruit and vegetable) as well as surfaces that may contact produce. Alternatively, a formulation that includes one or more sugar alcohols can be used as a solution or spray on or around produce.

In embodiments, the sugar alcohol in an anti-insect coating may be a sugar alcohol other than erythritol such as xylitol or mannitol. Additional sugar alcohols that may be employed to the same, or substantially the same molar ratios as the aforementioned sugar-alcohol constituents include sorbitol, ethylene glycol, glycerol, threitol, arabitol, xylitol, ribitol, mannitol, galactitol, fucitol, iditol, inositol, volemitol, isomalt, maltitol, lactitol, maltotriitol, maltotetraitol and/or polyglycitol.

In addition, the effects of other non-nutritive hexose and pentose sugar alcohols, such as sorbitol, mannitol, and xylitol, were tested. In aspects, an anti-insect formulation or coating includes about 1 M erythritol. In aspects, the formulation or coating includes about 2M erythritol and about 0.5M sucrose. In aspects, the formulation or coating does not include zinc.

### Skin and Scalp Infections

Skin infections of the scalp can be caused by bacteria or fungi entering through damaged skin or hair follicles. Some common types of scalp infections include folliculitis and tinea capitis. They are usually caused by staph bacteria, but can also be caused by other bacteria, parasites, or fungi. Symptoms include pimple-like sores around hair follicles, itching, tightness, and oozing or crusty sores. In severe cases, the infection can destroy hair follicles and cause permanent hair loss.

Tinea capitis (also known as ringworm of the scalp) is a fungal infection is caused by mold-like fungi and primarily affects children. Symptoms include itching, scaly, inflamed balding areas on the scalp. Oral antifungal medications are required to treat the infection. Other types of scalp infections include impetigo and lichen planus. A doctor can determine the cause of the infection and prescribe the appropriate treatment, which may include antibiotic lotions or pills, antifungal creams, shampoos, or pills.

Another embodiment is directed to a method of treating a skin or scalp infection (e.g., folliculitis and tinea capitis). The method can include steps of a) selecting a therapeutic agent (e.g., a sugar alcohol and/or zinc) described herein and formulating the therapeutic agent in a topical formulation, and b) administering the formulation topically and/or transdermally in an amount effective to treat the infection.

Another embodiment is directed to a method of preventing a skin or scalp infection that includes administering topically and/or transdermally an effective amount of a formulation that includes zinc and a sugar alcohol such as erythritol. Formulations provided herein can also be used in methods of treating other skin conditions that result from bacterial, fungal or yeast growth.

In another aspect, formulations of the invention can be administered or co-administered with one or more additional agents to treat a skin or scalp infection. For example, the topical formulation can be co-administered with an antiseptic (e.g., benzethonium chloride), an antibiotic or an anti-fungal (e.g., clotrimazole, econazole, miconazole, terbinafine, fluconazole, ketoconazole, nystatin or amphotericin).

### Acne Vulgaris

Acne, also known as acne vulgaris can be caused by bacteria, specifically the proliferation of Cutibacterium acnes, or C. acnes. Topical antibiotics (e.g., clindamycin and erythromycin) are common treatments. Oral antibiotics are used to treat acne but also have shortcomings as described above.

C. acnes is an ordinary resident of the skin, but in those with acne the population grows out of control. These bacteria irritate the skin's follicles, creating inflamed papules and pustules. Applying a topical antibiotic can reduce the amount of bacteria to help control acne. Topical antibiotics can also reduce inflammation, so they can be most effective with inflamed breakouts rather than non-inflamed blemishes or blackheads.

In an embodiment, a sugar alcohol is administered topically or transdermally to a subject. Although erythritol is described in the examples, other sugar alcohols can be used. Sugar alcohols include erythritol, xylitol, mannitol, sorbitol, ethylene glycol, glycerol, threitol, arabitol, xylitol, ribitol, mannitol, galactitol, fucitol, iditol, inositol, volemitol, isomalt, maltitol, lactitol, maltotriitol, maltotetraitol and polyglycitol. In an embodiment, a combination of sugar alcohols is administered to a subject to treat acne.

Another embodiment is directed to a method of treating acne. The method can include steps of a) selecting a therapeutic agent (e.g., a sugar alcohol and/or zinc) described herein and formulating the therapeutic agent in a topical formulation, and b) administering the formulation topically and/or transdermally in an amount effective to inhibit or prevent the growth and proliferation of C. acnes.

Another embodiment is directed to a method of preventing acne that includes administering topically and/or transdermally an effective amount of a formulation that includes a sugar alcohol such as erythritol and zinc. Formulations provided herein can also be used in methods of treating other skin conditions that result from bacteria growth such as cellulitis, erysipelas, bacterial folliculitis, hot tub folliculitis, furuncles, carbuncles, impetigo, erythrasma or MRSA skin infection.

In another aspect, formulations of the invention can be administered or co-administered with one or more additional agents that target acne. For example, the topical formulation can be co-administered with benzoyl peroxide, a retinoid, a steroid, an antibiotic, benzethonium chloride, azelaic acid, salicylic acid, dapsone, a contraceptive, an anti-androgen agent or isotretinoin to the subject.

In other aspects, formulations of the invention can be administered or co-administered with one or more additional agents that target biofilm. For example, the formulation of the present invention can be co-administered with other sugar alcohols, excipients and active agents without limiting the scope and disclosure of the present invention.

Although the examples describe the use of the zinc chloride-erythritol mixture to treat acne and prevent the propagation of biofilm, the formulations can be used to treat other ailments, including skin infections. For example, cellulitis, erysipelas, bacterial folliculitis, hot tub folliculitis, furuncles, carbuncles, impetigo, erythrasma and MRSA skin infection can be treated using the formulations described herein.

Embodiments include a topical lotion or cream for administration of an agent (e.g., erythritol) to a subject. It is placed on the skin to deliver a specific dose of an agent through the skin. The agent can be delivered across the skin into a localized subdermal location. In one embodiment, the lotion or cream includes one or more of a fragrance, a sunscreen, hyaluronic acid, an alpha-hydroxy acid (e.g., glycolic acid and lactic acid), a ceramide, retinol, argan oil, vitamin C, vitamin E, vitamin B3, green tea and algae extract.

An advantage of a transdermal drug delivery route over other types of delivery is that the formulation can provide a controlled release of the agent. Conventional transdermal delivery systems are generally ineffective for use with agents and medications that are large molecules and/or hydrophilic molecules.

There are other advantages to transdermal administration of medicaments. Small molecules can be inactivated or degraded by the stomach or liver. Transdermal administration is not affected by stomach or digestive issues. Further, people can benefit from drugs that are absorbed slowly and regularly. With a transdermal formulation, a medicament can be released in small quantities over a long period of time.

Other advantages are related to dosing. Large doses of agents can cause dose-dependent toxicity in many cases. For example, oral administration of vitamin A can result in hypervitaminosis A. The main problems associated with the oral administration of vitamin A are its half-life, fast absorption (due to lipophilicity) and its toxicity (due to high loading and frequent dosing). Also, some drugs undergo first-pass metabolism, which prevents their delivery to the desired site of action. Furthermore, many hydrophilic or lipophilic drugs show either poor dissolution or poor absorption on oral administration. With a topical or transdermal formulation, the effective concentration of an agent can be applied at the desired site without painful delivery.

In an embodiment, a topical formulation includes the components of Table 1:

TABLE 1
General Active Agent Formulation

| Ingredient | Weight (%) |
|---|---|
| Emollient/moisturizer | 10-20% |
| Alcohol | 0.5-2% |
| Oil | 1-5% |
| Surfactant | 0.5-2% |
| Deionized Water | 50-80% |
| Active Agent | 1-5% |
| Total | 100.00% |

In another embodiment, a topical formulation includes the components of Table 2 and the active agent (i.e., zinc chloride and erythritol) is 5% w/w:

TABLE 2
“A” Formulation- 5% active agent

| Ingredient | Weight (%) |
|---|---|
| A Humectant | 10% |
| Deionized Water | 83% |
| Poloxamer 407 | 2% |
| Zinc Chloride | 1.25% |
| Erythritol | 3.75% |
| Total | 100.00% |

In another embodiment, a topical formulation includes the components of Table 3 and the active agent (i.e., zinc chloride and erythritol) is 10% w/w:

TABLE 3
“B” Formulation- 10% active agent

| Ingredient | Weight (%) |
|---|---|
| A Humectant | 10% |
| Deionized Water | 78% |
| Poloxamer 407 | 2% |
| Zinc Chloride | 2.5% |
| Erythritol | 7.5% |
| Total | 100.00% |

In another embodiment, a topical formulation includes the components of Table 4 and the active agent (i.e., zinc chloride and erythritol) is 20% w/w:

TABLE 4
“B” Formulation- 20% active agent

| Ingredient | Weight (%) |
|---|---|
| A Humectant | 10% |
| Deionized Water | 68% |
| Poloxamer 407 | 2% |
| Zinc Chloride | 5% |
| Erythritol | 15% |
| Total | 100.00% |

In an aspect, the concentration of active agent is about 1%, about 1.5%, about 2%, about 3%, about 4%, about 5%, about 7.5%, about 12.5%, about 15%, about 17.5%, about 20%, about 25% or about 30%. In an aspect, the concentration of active agent is at least 0.5%, at least 1%, at least 1.5%, at least 2%, at least 7.5%, at least 12.5%, at least 15%, at least 17.5%, at least 20%, at least 25% or at least 30%. In an aspect, the concentration of active agent is not more than 0.5%, not more than 1%, not more than 1.5%, not more than 2%, not more than 7.5%, not more than 12.5%, not more than 15%, not more than 17.5%, not more than 20%, not more than 25% or not more than 30%.

In embodiments, the coating of the present invention improves a patient's skin condition (or skin appearance) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 100%.

In embodiments, the coating of the present invention reduces an inflamed and/or infected skin lesion surface area by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 100%.

In embodiments, the coating of the present invention reduces an inflamed and/or infected lesion surface area by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, about 100%.

In an embodiment, the concentration of deionized water in a transdermal formulation is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5% or more. In an embodiment, the concentration of Deionized Water in a transdermal formulation is about 0.1%, about 0.2%, about 0.3%, about 0.4%, about 0.5%, about 0.6%, about 0.7%, about 0.8%, about 0.9%, about 1%, about 2%, about 3%, about 4%, about 5% or more. In an embodiment, the concentration of deionized water in a transdermal formulation is from 0.1% to 5%, from 0.2% to 4%, from 0.3% to 3%, 0.4% to 2%, 0.5% to 1%, from 0.6% to 0.9%, from 0.7% to 0.8%, from 0.4% to 1.5%, from 0.3% to 0.7% or from 0.4% to 0.6%. In an embodiment, the concentration of deionized water in a formulation is no more than 0.1%, no more than 0.2%, no more than 0.3%, no more than 0.4%, no more than 0.5%, no more than 0.6%, no more than 0.7%, no more than 0.8%, no more than 0.9%, no more than 1%, no more than 2%, no more than 3%, no more than 4%, no more than 5% or more.

In an aspect, the transdermal and topical formulation also includes safflower oil in an amount of at least 1%, at least 2%, at least 3%, at least 4%, at least 5%, at least 7.5%, at least 10%, at least 11%, at least 11.06%, at least 12%, at least 13%, at least 14%, at least 15%, at least 16%, at least 17%, at least 18%, at least 19%, at least 20% or more. In an aspect, the concentration of Safflower oil in a formulation is about 1%, about 2%, about 3%, about 4%, about 5%, about 7.5%, about 10%, about 11%, about 11.06%, about 12%, about 13%, about 14%, about 15%, about 16%, about 17%, about 18%, about 19%, about 20% or more. In an aspect, the concentration of Safflower oil in a topical formulation is from 1% to 20%, from 5% to 19%, from 7.5% to 18%, from 10% to 17%, from 11% to 16%, from 11.06%, 12% from 11% to 12%, from 12% to 14%, from 13% to 14%, from 10% to 12%, from 10.5% to 12.5% or from 11% to 11.25%. In an aspect, the concentration of safflower oil in a topical formulation is no more than 1%, no more than 2%, no more than 3%, no more than 4%, no more than 5%, no more than 7.5%, no more than 10%, no more than 11%, no more than 11.06%, no more than 12%, no more than 13%, no more than 14%, no more than 15%, no more than 16%, no more than 17%, no more than 18%, no more than 19%, no more than 20%.

In a further aspect, the topical formulation includes oleic acid. The concentration of oleic acid can be, for example, at least 1%, at least 2%, at least 3%, at least 3.65%, at least 4%, at least 5%, at least 6%, at least 7%, at least 8%, at least 9%, at least 10% or more. In a further aspect, the concentration of oleic acid in a topical formulation is about 1%, about 2%, about 3%, about 3.65%, about 4%, about 5%, about 6%, about 7%, about 8%, about 9%, about 10% or more. In a further aspect, the concentration of oleic acid in a topical formulation is no more than 1%, no more than 2%, no more than 3%, no more than 3.65%, no more than 4%, no more than 5%, no more than 6%, no more than 7%, no more than 8%, no more than 9%, no more than 10% or more. In another aspect, the concentration of oleic acid in a transdermal formulation is from 1% to 10%, from 2% to 9%, from 2% to 3%, from 3% to 4%, from 3% to 8%, from 4% to 7%, from 5% to 6%, from 2 to 2.5% or from 2.5% to 4%.

In an aspect, the concentration of poloxamer 407 in a topical formulation is at least 10%, at least 15%, at least 20%, at least 25%, at least 28.75%, at least 30%, at least 35%, at least 40% or more. In an aspect, the concentration of poloxamer 407 in a topical formulation is not more than 10%, not more than 15%, not more than 20%, not more than 25%, not more than 28.75%, not more than 30%, not more than 35%, not more than 40% or more. In an aspect, the concentration of poloxamer 407 in a topical formulation is about 10%, about 15%, about 20%, about 25%, at least 28.75%, about 30%, about 35%, about 40% or more. In an aspect, the concentration of poloxamer 407 in a topical formulation is from 10% to 40%, is from 15% to 35%, is from 20% to 30%, is from 25% to 30%, is from 28% to 29%.

In an aspect, the concentration of poloxamer 407 in a medical device coating is at least 10%, at least 15%, at least 20%, at least 25%, at least 28.75%, at least 30%, at least 35%, at least 40% or more. In an aspect, the concentration of poloxamer 407 in a medical device coating is not more than 10%, not more than 15%, not more than 20%, not more than 25%, not more than 28.75%, not more than 30%, not more than 35%, not more than 40% or more. In an aspect, the concentration of poloxamer 407 in a medical device coating is about 10%, about 15%, about 20%, about 25%, at least 28.75%, about 30%, about 35%, about 40% or more. In an aspect, the concentration of poloxamer 407 in a medical device coating is from 10% to 40%, is from 15% to 35%, is from 20% to 30%, is from 25% to 30%, is from 28% to 29%.

In another aspect, the formulation includes glucose. The concentration of glucose in a topical formulation can be, for example, at least 1%, at least 2%, at least 2.5%, at least 3%, at least 4%, at least 5%, at least 6%, at least 7%, at least 8%, at least 9% or more. In another aspect, the concentration of glucose in a topical formulation is about 1%, about 2%, about 2.5%, about 3%, about 4%, about 5%, about 6%, about 7%, about 8%, about 9% or more. In another aspect, the concentration of glucose in a topical formulation is no more than 1%, no more than 2%, no more than 2.5%, no more than 3%, no more than 4%, no more than 5%, no more than 6%, no more than 7%, no more than 8%, no more than 9% or more. In another aspect, the concentration of glucose in a topical formulation is from 1% to 10%, is from 2% to 9%, is from 2.5% to 5%, is from 2% to 3%, is from 3% to 8%, if from 4% to 7%, if from 5% to 6%, is from 2% to 4%, is from 1.5% to 3.5%.

The formulation can also include penetrants including either or both chemical penetrants (CPEs) and peptide-based cellular penetrating agents (CPPs) that encourage transmission across the dermis and/or across membranes including cell membranes, as would be the case in particular for administration by suppository or intranasal administration, but for transdermal administration as well. In some embodiments, suitable penetrants include those that are described in the above-referenced US2009/0053290 ('290), WO2014/209910 ('910), and WO2017/127834. In addition to transdermal delivery formulations incorporating penetrants, transdermal delivery can be affected by mechanically disrupting the surface of the skin to encourage penetration, or simply by supplying the formulation applied to the skin under an occlusive patch.

The formulation can also include a gelling component. Suitable gelling components also include isopropyl palmitate, ethyl laurate, ethyl myristate and isopropyl myristate. The gelling agent can be less than 5% w/w of the formulation. In some embodiments, the formulation includes a mixture of xanthan gum, sclerotium gum, pullulan, or a combination thereof in an amount less than 2% w/w, 5% w/w, or 10% w/w of the formulation. In some embodiments, the formulation includes Siligel™ in an amount between about 1-5% w/w or 5-15% w/w, or an equivalent mixture of xanthan gum, sclerotium gum, and pullulan. In some embodiments, the formulation includes a mixture of caprylic triglycerides and capric triglycerides in amount less than 2% w/w, 8% w/w, or 10% w/w of the formulation. In some embodiments, the formulation includes Myritol® 312 in an amount between about 0.5-10% w/w, or an equivalent mixture of caprylic triglycerides and capric triglycerides.

An additional component in the present invention's formulations can be an alcohol. The weight percentage of benzyl or other related alcohol in the final composition can be 0.5-20% w/w, and again, intervening percentages such as 1% w/w, 2% w/w, 3% w/w, 4% w/w, 5% w/w, 6% w/w, 7% w/w, 8% w/w, 9% w/w, or 10% w/w, and other intermediate weight percentages are included. Due to the aromatic group present in a topical formulation such as benzyl alcohol, the molecule has a polar end (the alcohol end) and a non-polar end (the benzene end). This enables the agent to dissolve a wider variety of topical formulation components.

In some embodiments, the formulation includes a detergent portion in an amount between about 1-70% w/w or 1-60% w/w. In some embodiments, the nonionic detergent provides suitable handling properties whereby the formulations are gel-like or creams at room temperature. Suitable nonionic detergents include poloxamers such as the non-ionic surfactant Pluronic® and any other surfactant characterized by a combination of hydrophilic and hydrophobic moieties. Poloxamers are triblock copolymers of a central hydrophobic chain of polyoxypropylene flanked by two hydrophilic chains of polyethyleneoxide. Other nonionic surfactants include long chain alcohols and copolymers of hydrophilic and hydrophobic monomers where blocks of hydrophilic and hydrophobic portions are used.

In some embodiments, a topical formulation also contains surfactant, typically, nonionic surfactant at 2-25% w/w of a topical formulation along with a polar solvent wherein the polar solvent is present in an amount at least in molar excess of the nonionic surfactant. In these embodiments, typically, the composition comprises the above-referenced amounts of a topical formulation and benzyl alcohol along with a sufficient amount of a polar solution, typically an aqueous solution or polyethylene glycol solution that itself contains 10%-40% of surfactant, typically nonionic surfactant to bring the composition to 100%.

In some embodiments, a medical device coating also contains surfactant, typically, nonionic surfactant at 2-25% w/w of a topical formulation along with a polar solvent wherein the polar solvent is present in an amount at least in molar excess of the nonionic surfactant. In these embodiments, typically, the composition comprises the above-referenced amounts of a topical formulation and benzyl alcohol along with a sufficient amount of a polar solution, typically an aqueous solution or polyethylene glycol solution that itself contains 10%-40% of surfactant, typically nonionic surfactant to bring the composition to 100%.

In some embodiments other additives are included such as a gelling agent, a dispersing agent and a preservative. An example of a suitable gelling agent is hydroxypropylcellulose, which is generally available in grades from viscosities of from about 5 cps to about 25,000 cps such as about 1500 cps. All viscosity measurements are assumed to be made at room temperature unless otherwise stated. The concentration of hydroxypropylcellulose may range from about 1% w/w to about 2% w/w of the composition. Other gelling agents are known in the art and can be used in place of, or in addition to hydroxypropylcellulose. An example of a suitable dispersing agent is glycerin. Glycerin is typically included at a concentration from about 5% w/w to about 25% w/w of the composition. A preservative may be included at a concentration effective to inhibit microbial growth, ultraviolet light and/or oxygen-induced breakdown of composition components, and the like. When a preservative is included, it may range in concentration from about 0.01% w/w to about 1.5% w/w of the composition.

Additional components that can also be included in a topical formulation are fatty acids, terpenes, lipids, and cationic, and anionic detergents. In some embodiments, a topical formulation further comprises tranexamic acid in an amount less than 2% w/w, 5% w/w, or 10% w/w of the formulation. In some embodiments, a topical formulation further comprises a polar solvent in an amount less than 2% w/w, 5% w/w, 10% w/w, or 20% w/w of the transdermal delivery formulation. In some embodiments, a topical formulation further comprises a humectant, an emulsifier, an emollient, or a combination thereof. In some embodiments, a topical formulation further comprises almond oil in an amount less than about 5% w/w. In some embodiments, a formulation further comprises a mixture of thermoplastic polyurethane and polycarbonate in an amount less than about 5% w/w. In some embodiments, a topical formulation further comprises phosphatidylethanolamine in an amount less than about 5% w/w. In some embodiments, a topical formulation further comprises an inositol phosphatide in an amount less than about 5% w/w.

Other solvents and related compounds that can be used in some embodiments include acetamide and derivatives, acetone, n-alkanes (chain length between 7 and 16), alkanols, diols, short chain fatty acids, cyclohexyl-1,1-dimethylethanol, dimethyl acetamide, dimethyl formamide, ethanol, ethanol/d-limonene combination, 2-ethyl-1,3-hexanediol, ethoxydiglycol (Transcutol® by Gattefosse, Lyon, France), glycerol, glycols, lauryl chloride, limonene N-methylformamide, 2-phenylethanol, 3-phenyl-1-propanol, 3-phenyl-2-propen-1-ol, polyethylene glycol, polyoxyethylene sorbitan monoesters, polypropylene glycol 425, primary alcohols (tridecanol), 1,2-propane diol, butanediol, C₃-C₆ triols or their mixtures and a polar lipid compound selected from C₁₆ or C₁₈ monounsaturated alcohol, C₁₆ or C₁₈ branched saturated alcohol and their mixtures, propylene glycol, sorbitan monolaurate sold as Span® 20 by Sigma-Aldrich, squalene, triacetin, trichloroethanol, trifluoroethanol, trimethylene glycol and xylene.

Fatty alcohols, fatty acids, fatty esters, are bilayer fluidizers that can be used in some embodiments. Examples of suitable fatty alcohols include aliphatic alcohols, decanol, lauryl alcohol (dodecanol), unolenyl alcohol, nerolidol, 1-nonanol, n-octanol, and oleyl alcohol. Examples of suitable fatty acid esters include butyl acetate, cetyl lactate, decyl N,N-dimethylamino acetate, decyl N,N-dimethylamino isopropionate, diethyleneglycol oleate, diethyl sebacate, diethyl succinate, diisopropyl sebacate, dodecyl N,N-dimethyamino acetate, dodecyl (N,N-dimethylamino)-butyrate, dodecyl N,N-dimethylamino isopropionate, dodecyl 2-(dimethyamino) propionate, E0-5-oleyl ether, ethyl acetate, ethylaceto acetate, ethyl propionate, glycerol monoethers, glycerol monolaurate, glycerol monooleate, glycerol monolinoleate, isopropyl isostearate, isopropyl linoleate, isopropyl myristate, isopropyl myristate/fatty acid monoglyceride combination, isopropyl palmitate, methyl acetate, methyl caprate, methyl laurate, methyl propionate, methyl valerate, 1-monocaproyl glycerol, monoglycerides (medium chain length), nicotinic esters (benzyl), octyl acetate, octyl N,N-dimethylamino acetate, oleyl oleate, n-pentyl N-acetylprolinate, propylene glycol monolaurate, sorbitan dilaurate, sorbitan dioleate, sorbitan monolaurate, sorbitan monooleate, sorbitan trilaurate, sorbitan trioleate, sucrose coconut fatty ester mixtures, sucrose monolaurate, sucrose monooleate, tetradecyl N.N-dimethylamino acetate. Examples of suitable fatty acid-include alkanoic acids, caprid acid, diacid, ethyloctadecanoic acid, hexanoic acid, lactic acid, lauric acid, linoelaidic acid, linoleic acid, linolenic acid, neodecanoic acid, oleic acid, palmitic acid, pelargonic acid, propionic acid, and vaccenic acid. Examples of suitable fatty alcohol ethers include a-monoglyceryl ether, E0-2-oleyl ether, E0-5-oleyl ether, E0-10-oleyl ether, ether derivatives of polyglycerols and alcohols, and (1-O-dodecyl-3-O-methyl-2-O-2′,3′-dihydroxypropyl glycerol).

Examples of completing agents that can be used in some embodiments include β- and γ-cyclodextrin complexes, hydroxypropyl methylcellulose (e.g., Carbopol® 934), patchs, naphthalene diamide diimide, and naphthalene diester diimide.

One or more antioxidants can be included, such as vitamin C, vitamin E, proanthocyanidin and a-lipoic acid typically in concentrations of 0.1-2.5% w/w.

In some applications, it is desirable to adjust the pH of a topical formulation, compound or coating to assist in permeation or to adjust the nature of the target compounds in the subject or upon the applied apparatus. In some instances, the pH is adjusted to a level of pH 9-11 or 10-11 which can be done by providing appropriate buffers or simply adjusting the pH with base.

A topical formulation, compound and coating can include other components that act as excipients or serve purposes other than for treatment of acne. For example, preservatives like antioxidants (e.g., ascorbic acid or a-lipoic acid) and anti-inflammatory agents may be included. Other components apart from therapeutically active ingredients and components that are the primary effectors of dermal penetration may include those provided for aesthetic purposes such as menthol or other aromatics, and components that affect the physical state of the composition such as emulsifiers, for example, Durosoft®. Typically, these ingredients are present in very small percentages of the compositions. It is understood that these latter ancillary agents are neither therapeutic ingredients nor are they components that are primarily responsible for penetration of the skin. The components that primarily effect skin penetration have been detailed as described above. However, some of these substances have some capability for effecting skin penetration. See, for example, Kunta, J. R. et al, J. Pharm. Sci. (1997) 86:1369-1373, describing penetration properties of menthol.

The application method is determined by the nature of the treatment but may be less critical than the nature of the formulation itself. If the application is to a skin area, it may be helpful in some instances to prepare the skin by cleansing or exfoliation. In some instances, it is helpful to adjust the pH of the skin area prior to application of a topical formulation itself. The application of a topical formulation may be by simple massaging onto the skin or by use of devices such as syringes or pumps. Patches could also be used. In some cases, it is helpful to cover the area of application to prevent evaporation or loss of a transdermal delivery formulation.

Where the application area is essentially skin, it is helpful to seal-off the area of application subsequent to supplying a topical formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

In addition to the compositions and formulations of the invention per se, the methods can employ a subsequent treatment with linoleic acid. As transdermal treatments generally open up the skin barrier, which is, indeed, their purpose, it is useful to seal the area of application after the treatment is finished. Thus, treatment with a topical formulation may be followed by treating the skin area with a composition comprising linoleic acid to seal off the area of application. The application of linoleic acid is applicable to any transdermal procedure that results in impairing the ability of the skin to act as a protective layer. Indeed, most transdermal treatments have this effect as their function to allow the active component to pass through the epidermis to the dermis at least, and, if systemic administration is achieved, through the dermis itself.

Additional therapeutic agents can be included in the compositions. For example, hydrocortisone or hydrocortisone acetate may be included in an amount ranging from 0.25% w/w to about 0.5% w/w. Menthol, phenol, and terpenoids, e.g., camphor, can be incorporated for cooling pain relief. For example, menthol can be included in an amount ranging from about 0.1% w/w to about 1.0% w/w.

In some applications a formulation for transdermal delivery may, for example, comprise: Aveeno®, for example in an amount between about 10-95% w/w; between about 20-85% w/w, between about 20-75% w/w, between about 20-50% w/w.

The formulation described in this specification may also include more than one therapeutic compound as desired for the particular indication being treated, preferably those with complementary activities that do not adversely affect the other proteins. A topical formulation to be used for in vivo administration can be sterile. This can be accomplished, for instance, without limitation, by filtration through sterile filtration membranes, prior to, or following, preparation of a topical formulation or other methods known in the art, including without limitation, pasteurization.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

In certain embodiments, kits can include, without limitation, one or more creams or lotions comprising one or more formulations described herein. In various embodiments, the kit can comprise formulation components for transdermal, topical, or subcutaneous administration, formulated to be administered as an emulsion coated patch. In all of these embodiments and others, the kits can contain one or more lotion, cream, patch, disposable applicator or the like in accordance with any of the foregoing, wherein each patch contains a single unit dose for administration to a subject.

Imaging components can optionally be included, and the packaging also can include written or web-accessible instructions for using a transdermal delivery formulation. A container can include, for example, a vial, bottle, patch, syringe, pre-filled syringe, tube or any of a variety of formats well known in the art for multi-dispenser packaging.

In some embodiments, a suitable topical formulation includes: Siligel™ in an amount less than about 5% w/w; water in an amount between about 10-65% w/w; isopropyl palmitate in an amount between about 0.5-10% w/w; stearic acid in an amount between about 0.25-10% w/w; cetyl alcohol in an amount between about 0.25-10% w/w; glycerin in an amount between about 0.25-5% w/w; a topical formulation in an amount between about 0.25-10% w/w; ethanol in an amount less than about 5% w/w; benzyl alcohol in an amount less than about 5% w/w; sodium hydroxide 50% w/v in an amount between about 0.1-5% w/w; and sodium bicarbonate in an amount between about 1-32% w/w.

In some embodiments, a suitable medical device coating includes: Siligel™ in an amount less than about 5% w/w; water in an amount between about 10-65% w/w; isopropyl palmitate in an amount between about 0.5-10% w/w; stearic acid in an amount between about 0.25-10% w/w; cetyl alcohol in an amount between about 0.25-10% w/w; glycerin in an amount between about 0.25-5% w/w; a topical formulation in an amount between about 0.25-10% w/w; ethanol in an amount less than about 5% w/w; benzyl alcohol in an amount less than about 5% w/w; sodium hydroxide 50% w/v in an amount between about 0.1-5% w/w; and sodium bicarbonate in an amount between about 1-32% w/w.

In some embodiments, a suitable topical formulation includes Aveeno® in an amount between about 20-85% w/w; and sodium bicarbonate (3DF) in an amount between about 15-45% w/w.

In some embodiments, a topical formulation includes Aveeno® in an amount between about 20-85% w/w; and sodium bicarbonate in an amount between about 15-45% w/w.

The present formulations can include a nonionic surfactant. Applicant has found that by employing carbonate salts with particle sizes as disclosed herein, delivered with the penetrants as disclosed herein, and in some embodiments providing a combination of a nonionic surfactant and a polar gelling agent, the penetration capabilities of the carbonate salts of the resulting formulation and the effective level of delivery of the carbonate salts has been enhanced.

A formulation of the disclosure may be prepared in a number of ways. Typically, the components of a formulation are simply mixed together in the required amounts. Alternatively, some subset of these components can first be mixed and then “topped off” with the remaining components either simultaneously or sequentially. The precise manner of preparing a formulation will depend on the choice of carbonates and the percentages of the remaining components that are desirable with respect to that carbonate salt. In some embodiments, the water is in an amount between about 10-85% w/w, 15-50% w/w, or 15-45% w/w of the formulation.

The topical formulation is a multi-component mixture, whereby the particular concentrations of the penetration enhancers are informed in part by the molecular mass of the sodium bicarbonate, or sodium bicarbonate and the therapeutic agent to be transported. A topical formulation enables therapeutic agent to become bio-available to the target site within minutes of topical administration. A topical formulation permits the use of minimal concentrations of therapeutic agents, as little as 1/1000th of concentrations required of alternative processes, while enabling bioactivity and positive clinical outcomes simultaneously. In some embodiments, the topical formulation comprises an alcohol in an amount less than 5% w/w of the formulation.

### Administration and Dosing

A topical formulation provided herein can be topically administered in any form. For administration for the treatment of skin conditions a sufficient amount of the topical composition can be applied onto a desired area and surrounding skin, for example, in an amount sufficient to cover a desired skin surface. A topical formulation can be applied to any skin surface, including for example, facial skin, and the skin of the hands, neck, chest and/or scalp.

In applying a topical formulation of the invention, a topical formulation itself is simply placed on the skin and spread across the surface and/or massaged to aid in penetration. The amount of topical formulation used is typically sufficient to cover a desired surface area. In some embodiments, a protective cover is placed over the formulation once it is applied and left in place for a suitable amount of time, i.e., 5 minutes, 10 minutes, 20 minutes or more; in some embodiments an hour or two. The protective cover can simply be a bandage including a bandage supplied with a cover that is impermeable to moisture. This essentially locks in the contact of a topical formulation to the skin and prevents distortion of a topical formulation by evaporation in some cases. The composition may be applied to the skin using standard procedures for application such as a brush, a syringe, a gauze pad, a dropper, or any convenient applicator. More complex application methods, including the use of delivery devices, may also be used, but are not required. In an alternative to administering topically to intact skin, the surface of the skin may also be disrupted mechanically by the use of spring systems, laser powered systems, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed formulations that penetrate intact skin may be applied using occlusive patches, such as those in the form micro-patches. External reservoirs of the formulations for extended administration may also be employed.

In an alternative to administering topically to intact skin, the surface of the skin may also be disrupted mechanically by the use of spring systems, laser powered systems, use of iontophoresis, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed transdermal delivery formulations that penetrate intact skin may be applied using occlusive patches, such as those in the form of micro-patches. External reservoirs of the formulations for extended administration may also be employed.

Accordingly, in certain embodiments alternative methods of administering one or more therapeutic compounds or agents (e.g., medicaments) through intact skin are provided. As nonlimiting examples, these alternative methods might be selected from the following lists: on basis of working mechanism, spring systems, laser powered, energy-propelled, Lorentz force, gas/air propelled, shock wave (including ultrasound), on basis of type of load, liquid, powder, projectile, on basis of drug delivery mechanism, nano-patches, sandpaper (microdermabrasion), iontophoresis enabled, microneedles, on basis of site of delivery, intradermal, intramuscular, and subcutaneous injection. Other suitable delivery mechanisms include, without limitation, microneedle drug delivery, such as 3M Systems, Glide SDI (pushes drug as opposed to “firing” drug), MIT low pressure injectors, micropatches (single use particle insertion device), microelectro mechanical systems (MEMS), dermoelectroporation devices (DEP), transderm ionto system, TTS transdermal therapeutic systems, membrane-moderated systems (drug reservoir totally encapsulated in a shallow compartment), adhesive diffusion-controlled system (drug reservoir in a compartment fabricated from drug-impermeable metallic plastic backing), matrix dispersion type system (drug reservoir formed by homogeneously dispersing drug solids in a hydrophilic or lipophilic polymer matrix molder into medicated disc), and microreservoir system (combination of reservoir and matrix dispersion-type drug delivery system).

The application method is determined by the nature of the treatment but may be less critical than the nature of a topical formulation itself. If the application is to a skin area, it may be helpful in some instances to prepare the skin by cleansing or exfoliation. In some instances, it is helpful to adjust the pH of the skin area prior to application of the formulation itself. The application of a topical formulation may be by simple massaging onto the skin or by use of devices such as syringes or pumps. Patches could also be used. In some cases, it is helpful to cover the area of application to prevent evaporation or loss of the formulation.

Where the application area is essentially skin, it is helpful to seal-off the area of application subsequent to supplying a topical formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

A topical formulation can be applied in a single, one-time application, once a week, once a bi-week, once a month, or from one to twelve times daily, for a period of time sufficient to alleviate a condition, disease, disorder, symptoms, for example, for a period of time of one week, from 1 to 12 weeks or more, from 1 to 6 weeks, from 2 to 12 weeks, from 2 to 8 weeks, from 2 to 6 weeks, from 2 to 4 weeks, from 4 to 12 weeks, from 4 to 8 weeks, or from 4 to 6 weeks. The present compositions can be administered, for example, at a frequency of once per day to hourly if needed. The presently described formulations can be topically administered once or more per day for a period of time from 1 week to 4 weeks, of from 1 week to 2 weeks, for 1 week, for 2 weeks, for 3 weeks, or for 4 weeks or more. In some instances, it may also be desirable to continue treatment indefinitely, for example, to inhibit recurring inflammation. A suitable administration for a topical formulation comprising a skin cream, lotion or ointment, for example is once, twice, three, four times daily, or hourly if needed.

A coating can be applied in a single, one-time application, once a week, once a bi-week, once a month, or from one to twelve times daily, for a period of time sufficient to prevent the spread of biofilm, for example, for a period of time of one week, from 1 to 12 weeks or more, from 1 to 6 weeks, from 2 to 12 weeks, from 2 to 8 weeks, from 2 to 6 weeks, from 2 to 4 weeks, from 4 to 12 weeks, from 4 to 8 weeks, or from 4 to 6 weeks. The present compositions can be administered, for example, at a frequency of once per day to hourly if needed. The presently described formulations can be applied once or more per day for a period of time from 1 week to 4 weeks, of from 1 week to 2 weeks, for 1 week, for 2 weeks, for 3 weeks, or for 4 weeks or more. In some instances, it may also be desirable to continue treatment indefinitely, for example, to inhibit recurring inflammation. The coating may also be applied once, twice, three, four times daily, or hourly if needed.

As described above, if desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient or an application level for any particular surface will vary depending upon a variety of factors, including the activity of the specific active agent; the age, body weight, general health, sex and diet of the patient; the time of administration or application; the rate of excretion; possible drug combinations; the severity of the particular condition being treated; the area to be treated, the surface to be treated and the form of administration or application. One of ordinary skill in the art would appreciate the variability of such factors and would be able to establish specific dosing levels or amounts using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A topical formulation in accordance with the subject matter described herein may be a topical dosage form packaged in, for example, a multi-use or single-use package, including for example, a tube, a bottle, a pump, a container or bottle, a vial, a jar, a packet, a blister package, or any other disposable or reusable container.

Single dosage kits and packages containing a once per day amount of the topical formulation may be prepared. Single dose, unit dose, and once-daily disposable containers of the topical formulation are also provided.

Single coating application kits and packages containing a once per day amount of the coating may be prepared. Single dose, unit dose, and once-daily disposable containers of the coating are also provided.

The present formulation can remain stable in storage for periods including up to about 5 years, between about 3 months and about 5 years, between about 3 months and about 4 years, between about 3 months and about 3 years, and alternately any time period between about 6 months and about 3 years.

A formulation described herein remains stable for up to at least three years at a temperature of less than or equal to 40° C. In an embodiment, the presently described topical formulation remains stable for at least two years at a temperature of less than or equal to 40° C. In an embodiment, the presently described topical formulation remains stable for at least three years at a temperature of less than or equal to 40° C. and at a humidity of up to 75% RH, for at least two years at a temperature of less than or equal to 40° C. and at a humidity of up to 75% RH, or for at least three years at a temperature of less than or equal to 30° C. and at a humidity of up to 75% RH. In a further embodiment, the presently described topical formulation remains stable for an extended period of time when packaged in a multi-use container such as a bottle dispenser or the like, and exhibits equal to or even greater stability when packaged in a single-use package.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A topical formulation in accordance with the subject matter described herein may be a topical dosage form packaged in, for example, a multi-use or single-use package, including for example, a tube, a bottle, a pump, a container or bottle, a vial, a jar, a packet, or a blister package.

Dosing can be single dosage or cumulative (serial dosing), and can be readily determined by one skilled in the art. A topical formulation of the present invention may be administered once, twice, three, four, five, six, seven, eight, nine, ten, eleven, twelve, thirteen, fourteen, fifteen, sixteen, seventeen, eighteen, nineteen, twenty or more times to a subject. For instance, treatment of a disease may comprise a one-time administration of an effective dose of a topical formulation as disclosed herein. Alternatively, treatment of a disease may comprise multiple administrations of an effective dose of a topical formulation as carried out over a range of time periods, such as, e.g., once daily, twice daily, thrice daily, once every few days, or once weekly. The timing of administration can vary from individual to individual, depending upon such factors as the severity of an individual's symptoms. For example, an effective dose of a topical formulation as disclosed herein can be administered to an individual once daily for an indefinite period of time, or until the individual no longer requires therapy. A person of ordinary skill in the art will recognize that the condition of the individual can be monitored throughout the course of treatment and that the effective amount of a topical formulation disclosed herein that is administered can be adjusted accordingly. In one embodiment, a topical formulation as disclosed herein is capable of decreasing the time to resolve the symptoms of a disease, including in an individual suffering from a disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment.

In a further embodiment, a topical formulation and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In other embodiments, the coating and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months, five months, six months, one year, two years, three years, four years, five years or more.

In an embodiment, the period of administration of a topical formulation is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

The appropriate effective amount of a topical formulation disclosed herein to be administered to an individual can be determined by a person of ordinary skill in the art by taking into account factors, including, without limitation, an improvement in the individual based upon one or more clinical symptoms, and/or physiological indicators associated with improvements skin complexion, reduced number of comedones, papules and/or pustules, the particular characteristics, history and risk factors of the patient, such as, e.g., age, weight, general health and the like, or any combination thereof. Additionally, where repeated administration of a topical formulation is used, an effective amount of a topical formulation will further depend upon factors, including, without limitation, the frequency of administration, the half-life of the transdermal delivery formulation, or any combination thereof. It is known by a person of ordinary skill in the art that an effective amount of a topical formulation disclosed herein can be extrapolated from in vitro assays and in vivo administration studies using animal models prior to administration to humans or animals.

Wide variations in the necessary effective amount are to be expected in view of the differing efficiencies of the various routes of administration. For instance, oral administration of a topical formulation disclosed herein generally would be expected to require higher dosage levels than administration by inhalation. Similarly, systemic administration of a topical formulation disclosed herein would be expected to require higher dosage levels than a local administration. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors. One skilled in the art will recognize that the condition of the individual can be monitored throughout the course of therapy and that the effective amount of a therapeutic disclosed herein that is administered can be adjusted accordingly.

Aspects of the present specification disclose, in part, a reduction of the proliferation of biofilm. As used herein, the term “treating,” can refer to reduction of the accumulation of biofilm on a surface. For example, the term “treating” can mean reduction of biofilm on a surface by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual performance associated with antimicrobial medical device coatings are well known and can be determined by a person of ordinary skill in the art by using commonly known testing means and observations. Those of skill in the art will know the appropriate indicators associated with reduction of biofilm on a surface and will know how to determine if the surface has the reduced biofilm percentage disclosed herein.

In an embodiment, a first topical formulation is administered to an individual and at a later date, a second topical formulation is administered to the same individual.

In an embodiment, a first topical formulation is administered to an individual at the same time as a second topical formulation is administered to the individual.

In some embodiments, a component of the coating and formulation of the present invention may be glucose.

In some embodiments, the glucose is in an amount between about 0.05-10% w/w of the transdermal delivery formulation.

In some embodiments, the glucose is anhydrous dextrose in an amount between about 0.05-10% w/w of the transdermal delivery formulation.

In some embodiments, the formulation has a pH of 9-11.

In some embodiments, the formulation has a pH of 7-10.5.

In one embodiment, a topical formulation disclosed herein is capable of reducing the signs/symptoms associated with acne in an individual suffering from the acne by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, an anti-acne topical formulation is capable of reducing the number of comedones or microcomedones in an individual suffering from a acne by, e.g., about 10% to about 100%, about 20% to about 100%, about 30% to about 100%, about 40% to about 100%, about 50% to about 100%, about 60% to about 100%, about 70% to about 100%, about 80% to about 100%, about 90% to about 100%, about 10% to about 90%, about 20% to about 90%, about 30% to about 90%, about 40% to about 90%, about 50% to about 90%, about 60% to about 90%, about 70% to about 90%, about 80% to about 90%, about 10% to about 80%, about 20% to about 80%, about 30% to about 80%, about 40% to about 80%, about 50% to about 80%, or about 60% to about 80%, about 70% to about 80%, about 10% to about 70%, about 20% to about 70%, about 30% to about 70%, about 40% to about 70%, about 50% to about 70% or about 60% to about 70% as compared to a patient not receiving the same treatment.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Example 1

### Erythritol and Zinc to Treat Acne

In this example, an 18-year-old male visits a healthcare professional and complains of facial acne that has been persistent for the past three years. He describes sporadic facial breakouts that began gradually, varying in severity and never completely cleared. He explains that the breakouts contribute to feelings of low self-esteem. He tried over-the-counter (OTC) 5% benzoyl peroxide (BP) gels and washes, as well as multiple facial cleansing products, without improvement. A physical examination revealed a healthy teenager with 15 open and closed comedones, 10 papules, and 5 pustules on each half of the face and involving the forehead, cheeks and chin.

The healthcare professional administers the formulation of erythritol and zinc to the patient. As detailed in Table 2, the formulation includes erythritol (3.75%) and zinc chloride (1.25%). The formulation allows for effective transdermal administration of the zinc chloride-erythritol mixture which can prevent or ameliorate bacterial growth at or near skin pores and hair follicles.

The patient is also advised to continue daily use of an OTC facial wash. After washing and rinsing the face, the patient applies the formulation to the entire face. After five to ten minutes, the face can be rinsed with water. The routine is repeated each day (i.e., twice per day). Within ten days, the acne is resolved by about 80% (i.e., about 80% of the acne is gone). The comedones, papules and pustules are less apparent and only visible at close proximity. The patient resumes twice daily use of the formulation. Within two weeks the signs/symptoms of acne are resolved by about 95%. The patient has a clear complexion.

The patient is also advised to continue daily OTC facial washes and apply the transdermal formulation twice per week. The frequency can be increased if the patient observes a return of acne and/or a possible break out.

Transdermal administration allows direct absorption into a specific area. For example, a lotion can be applied to specific areas that are prone to acne. In this example, the patient applies the lotion to the forehead, cheeks and chin.

The lotion can include a topical formulation and the zinc chloride-erythritol mixture (collectively referred to as the formulation). In this example, the dose of the active agent (i.e., zinc chloride-erythritol mixture) is 5% w/w of the solution. A transdermal medicament presents several benefits. The lack of interference with food and alcohol is one advantage. Topical delivery avoids the GI tract and can increase bioavailability. Increased bioavailability permits lower doses which reduce the risk of side effects. Topical administration also allows for the patient to increase the volume and incidence of application based on need/symptoms.

### Example 2

Erythritol and Zinc with Exfoliator

In this example, a 16-year-old female visits a healthcare professional and complains of facial acne that become gradually worse over the past two years. She describes monthly and sporadic facial breakouts that never completely clear. She tried OTC benzoyl peroxide (BP) gels and washes, as well as multiple facial cleansing products, with minimal or no significant improvement.

The healthcare professional administers the transdermal formulation of erythritol and zinc to the patient. As detailed in Table 2, the formulation includes erythritol (3.75%) and zinc chloride (1.25%). The formulation allows for effective transdermal administration of the zinc chloride-erythritol mixture which can prevent or ameliorate bacterial growth at or near skin pores and hair follicles.

The patient is also advised to wash her face daily. After washing and rinsing the face, the patient applies the formulation to the entire face. After five to ten minutes, the face can be rinsed with water. This routine is repeated each day (i.e., twice per day). Within ten days, the acne is resolved by about 65% (i.e., about 65% of the acne is gone).

The healthcare professional administers a retinoid to be used in conjunction with the formulation. The retinoid cream can help unclog pores and increase absorption of the zinc chloride-erythritol mixture. Specifically, a pea-sized amount of retinoid cream is applied over the skin once a day 20 to 30 minutes after washing the face. Thereafter, the patent applies the formulation in a similar manner. Within two weeks the signs/symptoms of acne are resolved by about 95%. The patient has a clear complexion.

The patient is also advised to continue daily regular use of facial washes and to apply the retinoid and transdermal formulation twice per week. The frequency can be increased if the patient observes a return of acne and/or a possible break out.

### Example 3

Erythritol and Zinc with Steroid

In this example, a 17-year-old female visits a healthcare professional and complains of facial acne that has become gradually worse over the past three to five years. She describes monthly and sporadic facial breakouts that never completely clear. She tried OTC benzoyl peroxide (BP) gels and washes, as well as multiple facial cleansing products, with minimal or no significant improvement.

The healthcare professional administers the formulation of erythritol and zinc to the patient. As detailed in Table 2, the formulation includes erythritol (3.75%) and zinc chloride (1.25%). The formulation allows for effective administration of the zinc chloride-erythritol mixture.

The patient is also advised to wash her face daily. After washing and rinsing the face, the patient applies the formulation to the entire face. After five to ten minutes, the face can be rinsed with water. This routine is repeated each day (i.e., twice per day). Within ten days, the acne is resolved by about 65% (i.e., about 65% of the acne is gone). The healthcare professional observes mild inflammation in around the cheeks and forehead.

The healthcare professional administers hydrocortisone to be used in conjunction with the formulation. Topical hydrocortisone can reduce the inflammation of acne, and the swollen appearance that comes with it. Specifically, hydrocortisone cream is applied over the skin once a day 20 to 30 minutes after washing the face. Thereafter, the patent applies the formulation in a similar manner. Within two weeks the signs/symptoms of acne are resolved by about 95%. The patient has a clear complexion.

The patient is also advised to continue daily regular use of facial washes and to apply the hydrocortisone and transdermal formulation twice per week. The frequency can be increased if the patient observes a return of acne and/or a possible break out.

### Example 4

### Erythritol and Zinc for Treating an Eye Infection

There is increasing evidence that bacterial biofilms play a role in a variety of ocular infections. Bacterial growth is characterized as a biofilm when bacteria attach to a surface and/or to each other. This is distinguished from a planktonic or free-living mode of bacterial growth where these interactions are not present. Biofilm formation is a genetically controlled process in the life cycle of bacteria resulting in numerous changes in the cellular physiology of the organism, often including increased antibiotic resistance.

Topical administration of drugs to the eye for local delivery has been used successfully for years (e.g., eye drops for application directly to the eye or percutaneously absorptive compositions for passive diffusion across the skin or upper and/or lower eyelid). Conventional eye drops may be ineffective or have limitations associated with their inability to penetrate biofilm. In aspects, erythritol and zinc (at a molar ratio of about 3:1) are included in an antibiotic eye drop formulation. Their presence increases penetration of biofilm.

In this example, a 14-year-old male visits a healthcare professional and complains of itchy, red eyes. The symptoms have become gradually worse over the past two to three days. A health care practitioner suspects bacterial conjunctivitis which although a less frequent cause of conjunctivitis, is more common in children. The most common bacteria are Haemophilus influenza, Streptococcus pneumoniae and Staphylococcus aureus.

The practitioner provides an eye drop medication that includes a broad-spectrum topical antibiotic, specifically 0.5% chloramphenicol. The formulation also includes 6.6 mM zinc chloride and 19.8 mM erythritol (i.e., a molar ratio of 1:3). The drops are applied to the patient's eyes as follows: one to two drops every two hours for the first 24 hours, decreasing to six-hourly until the discharge resolves, for up to seven days.

The patient's symptoms decrease approximately 80% within 36 hours. Soon thereafter, the patient is asymptomatic. The patient is advised to continue using the eyedrops for a total of five days to ensure that the infection is resolved.

In an embodiment, an eye formulation includes the components of Table 5. The formulation can include an antibiotic (0.5-1% w/w). The formulation can also include a secondary active agent (i.e., zinc chloride and erythritol) at a concentration of 1-10% w/w.

TABLE 5
Eye Formulation

| Ingredient | Weight (%) |
|---|---|
| Deionized Water | 80-98% |
| Antibiotic | 0.5-1% |
| Secondary Active Agent | 1-10% |
| Inactive Ingredients | 0.25-10% |
| Total | 100.00% |

In aspects, the eye formulation includes 6.6 mM zinc chloride and 19.8 mM erythritol (i.e., a molar ratio of 1:3). The solution can also include trace amounts of salt and boric acid. The solution can also include tetrahydrozoline HCl (about 0.05% w/w). Inactive ingredients can include ascorbic acid, boric acid, dextrose, glycerin, glycine, magnesium chloride, polixetonium chloride, potassium chloride, sodium borate, sodium citrate, unspecified form, sodium lactate, sodium phosphate (dibasic, anhydrous). In aspects, the antibiotic is framycetin sulfate or chloramphenicol.

The eye formulation and methods described herein can be used to treat any eye infection that is associated with bacterial biofilms, including, conjunctivitis/pink eye, keratitis, endophthalmitis, blepharitis, sty, uveitis and cellulitis.

In embodiments, an eye formulation includes one active agent (i.e., zinc chloride and erythritol) at a concentration of 1-10% w/w (i.e., with no additional antibiotic). As described herein, a molar ratio of 1:3 (zinc chloride to erythritol) has demonstrated antimicrobial qualities.

### Example 5

### Erythritol and Zinc for Treating Eyes Incident to Surgery

Conventional eye drops may be ineffective or have limitations associated with their inability to penetrate biofilm. Conventional eye drops can also present unwanted side-effects. In this example, a 62-year-old male visits a healthcare professional for cataract surgery.

The health care practitioner provides the patient with an eye drop solution of erythritol and zinc (at a molar ratio of about 3:1) in sterile saline solution. The patient is advised to use the eye drops three days before surgery (e.g., three drops per eye, twice per day). After the surgery, the patient is advised to continue using the eye drops throughout the healing process (e.g., for an additional ten days).

The patient's vision improves as a result of the surgery. The eye drops prevent (or otherwise decrease the likelihood) of an eye infection incident to the surgery.

### Example 6

### Erythritol and Zinc as a Component of an Anti-Bacterial Coating

Colonization of bacteria on the surfaces of medical devices and healthcare products, particularly in implanted devices, result in serious patient problems, including the need to remove and/or replace the implanted device and to aggressively treat secondary infection conditions. The problem is due in part to biofilm—the thin, hard-to-treat layer of microorganisms that grows on hard surfaces and can result in serious infection.

In this example, a catheter is treated with a sterile solution of zinc chloride and erythritol (i.e., a molar ratio of 1:3). This method is a simple one-step soaking method which impregnates antimicrobials on the outer surface as well as inner lumen simultaneously.

In an embodiment, the sterile solution includes the components of Table 6. The formulation includes an active agent (i.e., zinc chloride and erythritol at a molar ratio of 1:3) at a concentration of 1-20% w/w.

TABLE 6
General Coating Formulation

| Ingredient | Weight (%) |
|---|---|
| Deionized Water | 80-98% |
| Active Agent | 1-20% |
| Inactive Ingredients | 0.25-10% |
| Total | 100.00% |

### Example 7

Erythritol and Zinc to Prevent Infection from Urinary Catheter

Urinary catheters are commonly used in patients with urological conditions. Following catheterization, bacteria attach to the catheter surface extraluminally (between the surface of the urinary tract and the catheter surface) and intraluminally, the latter originating through the drainage system and adhering to the catheter lumen surface. Once attached, bacteria then rapidly multiply and colonize on the surface of the device, producing bacterial biofilm. Unless the colonized catheter is removed, the biofilm has the potential to re-seed the bladder with microorganism, causing a urinary tract infection (UTI).

Unfortunately, existing antimicrobial catheters are expensive to produce due to the second coating of lubricious material such as hydrogel needed on the surface to offer comfort to the patients during insertion. Further, prolonged use of a catheter, such as a urinary catheter, or any medical device article, greatly increases the likelihood of inflammation or infection, such as catheter-associated urinary tract infection. These complications can be a major health concern, particularly in vulnerable populations such as the elderly, populations of developing countries, children and immunocompromised patients.

Biofilm associated problems experienced with implantable medical devices such as catheters, particularly catheters designed for urinary tract infections, pose a significant risk for catheterized patients of acquiring secondary infection such as nosocomial infection in a hospital environment. Such infections can result in prolonged hospital stay, administration of additional antibiotics, and increased cost of post-operative hospital care. In biofilm mediated urinary tract infections, bacteria are believed to gain access to the catheterized bladder either by migration from the collection bag, the catheter by adhering to and proliferating on the material constituting the catheter material, or by ascending the periurethral space outside the catheter.

In this example, a patient uses a “post-operative” intermittent urinary catheter. The catheter is inserted several times a day, for just long enough to drain the patient's bladder, and then removed. As described below, the catheter is cleaned and sanitized for re-use on a patient. The catheter is rinsed with an “anti-biofilm” solution containing 6.6 mM zinc chloride and 19.8 mM erythritol. The solution can also include a lubricious material (e.g., a hydrogel). This step can prevent and/or ameliorate the formation of biofilm on the lumen of the catheter.

Intermittent bladder catheterization is done for a variety of urinary tract problems. It is generally done using “clean technique.” This means that not all bacteria are kept from coming into contact with the person being catheterized. A health care provider explains the following steps to the patient:
a) Clean the catheter by rinsing with tap or distilled water for 30 seconds.
b) Place the catheter in anti-biofilm solution (6.6 mM zinc chloride and 19.8 mM erythritol) for one to five minutes.
c) Remove the catheter from the anti-biofilm solution and place in a freezer bag.
d) Store the catheter in a freezer bag without rinsing.

The catheter does not have to be rinsed with water before use. The cleaning step can include a step of sanitizing the catheter with alcohol (e.g., 70% EtOH) before it is rinsed with water and placed into the anti-biofilm solution.

### Example 8

Erythritol and Zinc Administered in Conjunction with Microdermabrasion

Microdermabrasion is a process for removing dead cells from the outermost layer of the skin (i.e., the epidermis) which can provide a younger and healthier looking appearance, remove wrinkles, clean out blocked pores, alleviate some types of undesirable skin conditions and enhance skin tone. Crystal microdermabrasion uses force or friction to remove the epidermis, while diamond microdermabrasion is more abrasive, sanding off the top layer of skin.

A 35-year-old male who has been battling acne on-and-off for years and decides to seek alternative acne treatment from the oral antibiotics and ointments which have been ineffective. In this example, the patient receives crystal microdermabrasion. The practitioner targets areas of the skin with the acne pustules including the patient's face and back. After the treatment, a practitioner applies the moisturizing formulation directly to the skin. The moisturizing formulation includes 5% w/w of zinc-erythritol (1:3 molar ratio).

The patient is advised to use OTC cleansers on his body and apply the moisturizing formulation daily. After three weeks (and one treatment session), the patient reports an 85% reduction in acne pustules. Treatments thereafter are continued once per month or as needed. After 12 weeks of treatment, the patient reports that none of the eliminated acne pustules have returned, and that his signs/symptoms of acne have been reduced by at least 95%.

### Example 9

Erythritol and Zinc Administered in Conjunction with Hydrafacial®

Microdermabrasion is a process for removing dead cells from the outermost layer of the skin (i.e., the epidermis). Similarly, hydrafacial methods involve cleansing and exfoliation of the skin. A healthcare professional can uses a wand to suck out and extract unwanted impurities and blackheads from the skin. After this step, a deep serum can be applied to the skin (with moisturizer, antioxidants, etc.) to hydrate and soften the skin. This method can stimulate production of collagen and improve one's complexion while treating undesirable skin problems.

In this example, a 22-year-old male with noticeable acne seeks treatment from a health professional. Previous use of OTC acne treatments, oral antibiotics and steroid ointments have been ineffective. The patient receives a transdermal treatment using a HydraFacial® Tower™. The practitioner then applies a “peel” to help exfoliate the skin and remove debris. A vacuum attachment is used to further clean the skin surface before applying a skin formulation. The formulation includes zinc-erythritol and is administered by a handheld device with an applicator tip.

The patient is advised to use OTC detergents and apply a moisturizer (that includes zinc-erythritol) daily. Two weeks after treatment, the patient reports a 90% reduction in acne pustules. Treatments thereafter are continued once per month or as needed.

### Example 10

Erythritol and Zinc Administered in Conjunction with Hydrafacial®

In this example, a 17-year-old male with heavy pustules approaches a healthcare professional for advice. Specifically, he seeks an alternative acne treatment because oral antibiotics and ointments have been ineffective. The healthcare professional recommends microdermabrasion in combination with topical erythritol-zinc treatment. The patient receives a single transdermal treatment that includes a nourishing serum with a zinc-erythritol formulation as described herein (“Formulation A”) administered by a handheld device with an applicator tip (e.g., a Hydrafacial® machine). The device simultaneously exfoliates the skin and applies the formulation to the skin surface. After treatment, the patient is administered an OTC facial wash formulated for oily skin.

Within one week treatment, the patient reports an 85% reduction in acne pustules. Treatments thereafter are continued every six weeks or as needed. After the second treatment, the patient reports that none of the eliminated acne pustules have returned, and that his signs/symptoms of acne have been reduced by at least 95%.

### Example 11

Erythritol and Zinc Administered in Conjunction with Hydrafacial®

Hydradermabrasion is a relatively new procedure that combines crystal-free microdermabrasion with the pneumatic application of an antioxidant-based serum. In this example, a 58-year-old female contacts a cosmetic dermatologist seeking to improve the appearance of her skin on her face and neck. The patient has used conventional exfoliants and moisturizing agents daily. However, she seeks a more aggressive treatment without resorting to a chemical face peel or surgery. The dermatologist recommends HydraFacial® or hydradermabrasion to remove fine wrinkles, reduce redness/inflammation and improve the glow/outward appearance. The method involves a three-step regimen that includes cleansing, exfoliating, and infusing the skin with nourishing serums.

The patient receives a single transdermal treatment that includes a nourishing serum with a zinc-erythritol formulation as described herein administered by a handheld device with an applicator tip (e.g., a Hydrafacial® machine). The device simultaneously exfoliates the skin, clears debris and applies the formulation to the skin surface. The formulation includes the components of Table 7 below.

After the initial treatment, the patient notes a significant improvement in the appearance of her skin. Treatments thereafter are continued every four weeks. The patient enjoys thicker keratinous tissue, less red blotchiness, improved skin texture (e.g., less wrinkles and fine lines) and more radiant skin.

TABLE 7
Microdermabrasion Formulation

| Ingredient | Weight (%) |
|---|---|
| Emollient/moisturizer | 15% |
| Alcohol | 2% |
| Oil | 3% |
| Surfactant | 2% |
| Deionized Water | 73% |
| Erythritol | 3.75% |
| Zinc Chloride | 1.25% |
| Total | 100.00% |

### Example 12

### Use of Erythritol and Zinc in a Dermal Filler

Infection can occur immediately or some weeks after a medical cosmetic procedure such as an injection. A patient may experience redness, tenderness, swelling and pain with or without nodules. A zinc-erythritol solution can be used to reduce the risk of infection associated with a treatment. The zinc-erythritol can also reduce the risk of formation of granulomas.

Dermal fillers are soft, gel-like substances that are injected under the skin. They are used to improve one's appearance (i.e., cosmetically) and can smooth under-eye circles, lift the cheekbones, volumize the lips, smooth lip lines and nasolabilal folds (the creases that run from the side of the nose to the corners of the mouth), and rejuvenate the hands. Dermal fillers can be composed of a variety of substances, some naturally occurring and some synthetic. One of the most common compounds used in dermal fillers is hyaluronic acid (HA). Some formulations can be dissolved by injecting a second solution in case of an adverse event. HA fillers can also be premixed with lidocaine to reduce pain during the procedure.

Other components of dermal fillers include calcium hydroxylapatite, poly-L-lactic acid, polymethyl methacrylate, and autologous fat (fat that is transplanted from another part of your body). Calcium hydroxylapatite is a mineral-like compound naturally found in human bones. Poly-L-lactic acid is a synthetic filler that helps to stimulate collagen production. Polymethyl methacrylate is a semi-permanent filler though it can cause potential complications such as forming visible lumps under the skin.

Known side effects reported in clinical trials include facial weakness, eyelid drooping, and brow drooping. Other adverse events included localized pain, swelling, reddening, and bruising at the injection site. Infections secondary to HA fillers occasionally occur due to violation of the protective skin barrier, whether during injection or in the post-treatment period. Acute infections occur within two weeks of treatment and present with inflammation and/or abscess at the site of injection.

In this example, a 62-year-old female contacts a cosmetic dermatologist seeking to improve the appearance of her skin on her face and neck. The patient wishes to avoid techniques such as chemical face peel or surgery. The dermatologist recommends a dermal filler to remove fine wrinkles and add volume to the lips/neck/cheeks. The patient receives a single transdermal treatment that includes injection of a hyaluronic acid dermal filler. The filler also includes 2.5% w/w of a 3:1 ratio of erythritol to zinc chloride.

The procedure includes the following steps:
1) Facial assessment & mapping: The health care professional evaluates the patient's facial appearance and skin tone and examines the areas of your face to be augmented.
2) Strategic points on your face may be marked as the appropriate injection sites for the filler. Photographs may be taken of the areas to be treated.
3) Cleansing and anesthetizing: The injection sites are cleansed with an antibacterial agent. Pain at the injection site may be ameliorated by use of a very cold instrument to chill the skin, anesthetic ointment to numb the skin or injection of local anesthesia.
4) Injection: Injection usually takes only a few moments per site. The process of injecting, massaging, and evaluating the result is performed, and additional filler added as needed.

After the treatment, the patient notes a significant improvement in the appearance of her skin. Treatments thereafter are continued once every six months. The patient enjoys thicker keratinous tissue and improved skin texture (e.g., less wrinkles and fine lines).

### Example 13

### Erythritol and Zinc for Treating Dandruff

Dandruff (i.e., seborrheic dermatitis) is a common skin condition that mainly affects the scalp. The main symptoms of dandruff are an itchy scalp and flakiness. Red and greasy patches of skin and a tingly feeling on the skin are also symptoms. A more severe form of the condition, which includes inflammation of the skin, is known as seborrhoeic dermatitis.

The cause of dandruff may involve a number of genetic and environmental factors. Conventional treatments include antifungal cream (e.g., ketoconazole) or the keratolytic agent salicylic acid may be used to try to improve the condition.

In this example, a teenage male visits a dermatologist with flaking and itchiness in and around the scalp. Upon examination, the physician notes patches of inflammation (i.e., redness).

The patient receives a shampoo for daily use that includes 5% of a zinc-erythritol formulation as described herein. The shampoo also includes 0.2% benzethonium chloride (BTC). After five days of use, the patient notes a significant improvement in the appearance of his scalp and hair. The dandruff is reduced by more than 80% and the red patches are absent.

### Example 14

### Body Wash Containing Erythritol and Zinc

In this example, a teenage male consults a healthcare provider for advice of hygiene. Specifically, the patient desires a body wash to ameliorate body odor. However, he does not want to use products with strong scents, perfumes nor harsh cleansers.

The patient receives a body wash for daily use. The base formula includes water and botanical hydrosols, surfactants to cleanse the skin, a preservative to protect against microbes and a thickener to increase the viscosity of the product. The body wash also includes 5% of a zinc-erythritol formulation as described herein (i.e., erythritol and zinc at a molar ratio of about 3:1).

After two daily uses, the patient notes a significant reduction in body odor. The patient is particularly pleased that the body wash is gentle on his skin. Further, the patient no longer relies on deodorant sticks and colognes to reduce/avoid body odor.

### Example 15

Erythritol and Zinc Administered in Conjunction with Antibiotics

Fungal infections are common in humans and are usually not very serious if they are treated quickly and correctly. Anyone with a weakened immune system may be more likely to contract a fungal infection, as well as anyone who is taking antibiotics.

In this example, a female patient with a history of recurrent yeast infections receives a broad-spectrum oral antibiotic to treat a respiratory infection. Because the patient is susceptible to yeast infection, her healthcare provider recommends daily use of a topical lotion to ameliorate yeast growth. The lotion includes the components of Table 8 below.

TABLE 8
Microdermabrasion Formulation

| Ingredient | Weight (%) |
|---|---|
| Emollient/moisturizer | 15% |
| Alcohol | 1.8% |
| Oil | 3% |
| Surfactant | 2% |
| Deionized Water | 73% |
| Erythritol | 3.75% |
| Zinc Chloride | 1.25% |
| Benzethonium Chloride | 0.2% |
| Total | 100.00% |

Daily use of the lotion prevents yeast growth. The patient does not experience further yeast infections despite the use of the antibiotics. The healthcare provider advises her to use the lotion if she notices any signs/symptoms of yeast infection (e.g., itchiness, redness, discharge, etc.).

### Example 15

### Anti-Yeast Formulation of Erythritol and Zinc

Benzethonium chloride (BTC) also known as hyamine is a synthetic quaternary ammonium salt. This compound is an odorless white solid, soluble in water. It has surfactant, antiseptic, and anti-infective properties, and it is used as a topical antimicrobial agent in first aid antiseptics. It is also found in cosmetics and toiletries such as soap, mouthwashes, anti-itch ointments, and antibacterial moist towelettes. Benzethonium chloride is also used in the food industry as a hard surface disinfectant.

BTC exhibits a broad spectrum of microbiocidal activity against bacteria, fungi, mold and viruses. Studies have demonstrated that benzethonium chloride is effective against such pathogens as methicillin-resistant Staphylococcus aureus, Salmonella, Escherichia coli, Clostridium difficile, hepatitis B virus, hepatitis C virus, herpes simplex virus (HSV), human immunodeficiency virus (HIV), respiratory syncytial virus (RSV), and norovirus.

In this example, a disinfecting wipe includes zinc chloride and erythritol (i.e., a molar ratio of 1:3) along with benzethonium chloride. The disinfecting wipes are used clinically on surfaces to reduce the incidence of bacterial and yeast infection. The wipes can also be used directly on the surface of the skin.

In an embodiment, the wipes are provided in a solution that includes the components of Table 9. The formulation can include an active agent (i.e., zinc chloride and erythritol at a molar ratio of 1:3) at a concentration of 1-20% w/w. Additionally, benzethonium chloride (BTC) can be included at 0.1 to 1.0% w/w.

TABLE 6
General Coating Formulation

| Ingredient | Weight (%) |
|---|---|
| Deionized Water | 80-98% |
| Active Agent | 1-20% |
| Inactive Ingredients | 0.25-10% |
| Total | 100.00% |

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s).

Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method of preventing or treating a skin or scalp condition, the method comprising applying a therapeutic amount of a topical formulation comprising erythritol and zinc to the skin or scalp,
wherein the erythritol and zinc are at a molar ratio of about 3:1.

2. The method of claim 1, wherein the skin or scalp condition is caused by yeast.

3. The method of claim 1, wherein the skin or scalp condition is caused by bacteria.

4. The method of claim 1, wherein the skin or scalp condition is selected from folliculitis, dandruff, seborrheic dermatitis, athlete's foot, yeast infection, jock itch/Tinea cruris, a toe nail infection, diaper rash and ringworm.

5. The method of claim 1, wherein the skin or scalp condition is selected from athlete's foot, a yeast infection, jock itch (Tinea cruris), a toe nail infection, diaper rash, diaper candidiasis, ringworm (Tinea capitis), impetigo, erythrasma, pitted keratolysis, trichomycosis and Sphingomonas paucimobilis.

6. The method of claim 1, wherein the topical formulation is applied as a spray, a cream, a roll on, a body wash a shampoo or a soap.

7. The method of claim 1, wherein the topical formulation comprises about 19.8 mM erythritol and about 6.6 mM of zinc.

8. The method of claim 1, further comprising a step of applying an additional antibacterial or antifungal medicament to the skin or scalp.

9. The method of claim 1, wherein the formulation further comprises benzethonium chloride (BTC).

10. A method of preventing or ameliorating body odor (bromhidrosis), the method comprising applying a therapeutic amount of a formulation comprising erythritol and zinc to one or more regions of skin,
wherein the erythritol and zinc are at a molar ratio of about 3:1.

11. The method of claim 10, wherein the body odor is caused by bacteria.

12. The method of claim 10, wherein the body odor is caused by yeast.

13. The method of claim 10, wherein the formulation is applied as a spray, a cream, a roll on, a body wash a shampoo or a soap.

14. The method of claim 10, wherein the formulation comprises about 19.8 mM erythritol and about 6.6 mM of zinc.

15. The method of claim 10, wherein the formulation further comprises one or more of benzethonium chloride, a fragrance, an emollient, a moisturizer, an alcohol, an oil, a surfactant and a humectant.

16. An antimicrobial solution or coating for a personal care product, the coating comprised of erythritol and zinc, wherein the erythritol and zinc are at a molar ratio of about 3:1.

17. The antimicrobial solution or coating of claim 16, wherein the personal care product is a diaper, a sanitary wipe, a wet wipe, a sanitary pad or a tampon.

18. The antimicrobial solution or coating of claim 16, wherein the solution or coating further comprises one or more of a fragrance, an emollient, a moisturizer, an alcohol, an oil, a surfactant and a humectant.

19. The antimicrobial solution or coating of claim 16, wherein the solution or coating is further comprised of benzethonium chloride (BTC).

20. The antimicrobial solution or coating of claim 16, wherein the personal care product is an intimate cleanser or body wipe.
